(19) United States
(12) Patent Application Publication
(10) Pub. No.: US 2022/0387562 A1
(43) Pub. Date: Dec. 8, 2022

(54) COMPOSITIONS AND METHODS FOR TREATING GLYCOGEN STORAGE DISORDERS

(71) Applicant: Audentes Therapeutics, Inc., San Francisco, CA (US)

(72) Inventors: John T. GRAY, Boston, MA (US); Justine CUNNINGHAM, Irvine, CA (US); Salvador RICO, Berkeley, CA (US)

(21) Appl. No.: 17/771,627

(22) PCT Filed: Oct. 23, 2020
(86) PCT No.: PCT/US20/57081

§ 371 Date: Apr. 25, 2022

Related U.S. Application Data
(60) Provisional application No. 62/926,282, filed on Oct. 25, 2019.
(60) Provisional application No. 63/083,349, filed on Sep. 25, 2020.

(51) Int. Cl. A61K 38/47; A61K 48/00; A61P 3/00

(52) U.S. Cl. CPC A61K 38/47; A61K 48/0025; A61K 48/0075; A61K 48/0058; A61P 3/00

(57) ABSTRACT

The present disclosure relates to compositions and methods useful for treating glycogen storage disorders, such as type II glycogen storage disorder, also referred to herein as Pompe disease. Using the compositions and methods of the disclosure, a patient (e.g., a mammalian patient, such as a human patient) having Pompe disease may be administered a viral vector, such as an adeno-associated viral (AAV) vector, that contains a transgene encoding acid alpha-glucosidase.

### FIELD OF THE INVENTION

The present disclosure relates to the field of gene therapy and provides compositions and methods for ameliorating genetic disorders.

### SEQUENCE LISTING

The instant application contains a Sequence Listing which has been submitted electronically in ASCII format and is hereby incorporated by reference in its entirety. Said ASCII copy, created on Oct. 23, 2020 is named 51037-054WO3_Sequence_Listing_10.23.20_ST25 and is 13,144 bytes in size.

### BACKGROUND OF THE INVENTION

Pompe disease is a lysosomal storage disorder caused by mutations in the acid alpha-glucosidase (GAA) gene, which encodes an enzyme responsible for processing lysosomal glycogen. Patients with Pompe disease exhibit clinical phenotypes across a variety of tissues, including glycogen buildup in cells, deficits in cardiac, respiratory, and skeletal muscle function, and central nervous system pathology. Some of these deficits are significantly ameliorated by enzyme replacement therapy (ERT) using recombinant human GAA (rhGAA). Clinical efficacy has been limited by the immunogenicity of hGAA ERT and the lack of uptake of rhGAA into some affected tissues. Gene therapy has also been investigated as a potential therapeutic paradigm for this disease. The development of gene therapies for the treatment of Pompe have been hindered by the difficulty associated with achieving expression of therapeutically effective amounts of GAA in affected tissues while suppressing toxic side effects. There remains a need for compositions and methods that achieve this balance.

### SUMMARY OF THE INVENTION

The present disclosure provides compositions and methods that can be used for treating glycogen storage disorders, such as type II glycogen storage disorder, which is also referred to herein as Pompe disease. Using the compositions and methods of the disclosure, a patient (e.g., a mammalian patient, such as a human patient) having Pompe disease may be administered a viral vector, such as an adeno-associated viral (AAV) vector, that contains a transgene encoding acid alpha-glucosidase (GAA). The AAV vector may be, for example, a pseudotyped AAV vector, such as an AAV vector containing AAV2 inverted terminal repeats packaged within capsid proteins from AAV8 (AAV2/8) or AAV9 (AAV2/9). The transgene may, for example, be operably linked to a transcription regulatory element, such as a promoter that induces gene expression in a muscle cell and/or a neuronal cell. Exemplary promoters that may be used in conjunction with the compositions and methods of the disclosure are a muscle creatine kinase promoter, desmin promoter, and CMV promoter, among others. The AAV vector may be administered to the patient in a therapeutically effective amount, such as in an amount of from about 1×10¹³ vector genomes (vg) per kg of body weight of the subject (vg/kg) to about 3×10¹⁴ vg/kg (e.g., in an amount of from about 3×10¹³ vg/kg to about 2×10¹⁴ vg/kg, such as in an amount of from about 4×10¹³ vg/kg to about 1×10¹⁴ vg/kg, such as in an amount of about 4×10¹³ vg/kg, 5×10¹³ vg/kg, 6×10¹³ vg/kg, 7×10¹³ vg/kg, 8×10¹³ vg/kg, 9×10¹³ vg/kg, or 1×10¹⁴ vg/kg).

The present disclosure is based, in part, on the discovery of doses of AAV vectors containing a GAA transgene that effectuate a therapeutic increase in GAA expression and activity in patients suffering from Pompe disease while suppressing toxic side effects. It has presently been discovered, for example, that doses of AAV vectors containing a transgene encoding GAA ranging from about 1×10¹³ vg/kg to about 3×10¹⁴ vg/kg (e.g., from about 3×10¹³ vg/kg to about 2×10¹⁴ vg/kg, such as a dose of about 4×10¹³ vg/kg, 5×10¹³ vg/kg, 6×10¹³ vg/kg, 7×10¹³ vg/kg, 8×10¹³ vg/kg, 9×10¹³ vg/kg, or 1×10¹⁴ vg/kg) can engender a beneficial increase in GAA expression and activity in a patient having Pompe disease while simultaneously avoiding toxic side effects that can be associated with overexpression of GAA or administration of excessive quantities of viral vector. Using the compositions and methods of the disclosure, an AAV vector may be administered to the patient in an amount that is sufficient to enhance the patient's expression of GAA and reduce cellular accumulation of glycogen in the patient's neuronal and muscle tissue, without inducing toxic side effects.

In a first aspect, the disclosure features a method of treating Pompe disease in a human patient in need thereof by administering to the patient an AAV vector containing a transgene encoding acid GAA, wherein the AAV vector is administered to the patient in an amount of from about 1×10¹³ vg/kg to about 5×10¹⁴ vg/kg, such as in an amount of from about 1×10¹³ vg/kg to about 3×10¹⁴ vg/kg. For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a method of improving muscle function in a human patient diagnosed as having Pompe disease by administering to the patient an AAV vector containing a transgene encoding GAA, wherein the AAV vector is administered to the patient in an amount of from about 1×10¹³ vg/kg to about 5×10¹⁴ vg/kg, such as in an amount of from about 1×10¹³ vg/kg to about 3×10¹⁴ vg/kg. For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In a further aspect, the disclosure features a method of reducing glycogen accumulation in a human patient diagnosed as having Pompe disease by administering to the patient an AAV vector containing a transgene encoding GAA, wherein the AAV vector is administered to the patient in an amount of from about 1×10¹³ vg/kg to about 5×10¹⁴ vg/kg, such as in an amount of from about 1×10¹³ vg/kg to about 3×10¹⁴ vg/kg. For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg. In some embodiments of this aspect, administration of the AAV vector to the patient reduces glycogen accumulation in muscle tissue (e.g., in cardiac and/or skeletal muscle tissue) and/or in neuronal tissue.

In another aspect, the disclosure features a method of improving pulmonary function in a human patient diagnosed as having Pompe disease by administering to the patient an AAV vector containing a transgene encoding GAA, wherein the AAV vector is administered to the patient in an amount of from about 1×10¹³ vg/kg to about 5×10¹⁴ vg/kg, such as in an amount of from about 1×10¹³ vg/kg to about 3×10¹⁴ vg/kg. For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a method of increasing GAA expression in a human patient diagnosed as having Pompe disease by administering to the patient an AAV vector containing a transgene encoding GAA, wherein the AAV vector is administered to the patient in an amount of from about 1×10¹³ vg/kg to about 5×10¹⁴ vg/kg, such as in an amount of from about 1×10¹³ vg/kg to about 3×10¹⁴ vg/kg. For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In some embodiments of any of the above aspects of the disclosure, the AAV vector is administered to the patient in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the AAV vector is administered to the patient in an amount of from about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

In some embodiments of any of the above aspects of the disclosure, the AAV vector is administered to the patient in an amount of from about 3×10¹³ vg/kg to about 2×10¹⁴ vg/kg, such as an in amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of from about 4×10¹³ vg/kg to about 2×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of from about 5×10¹³ vg/kg to about 2×10¹⁴ vg/kg, such as an in amount of about 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of from about 6×10¹³ vg/kg to about 2×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of from about 7×10¹³ vg/kg to about 2×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of from about 8×10¹³ vg/kg to about 2×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of from about 9×10¹³ vg/kg to about 2×10¹⁴ vg/kg, such as an in amount of 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of from about 1×10¹⁴ vg/kg to about 2×10¹⁴ vg/kg, such as an in amount 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the above aspects of the disclosure, the AAV vector is administered to the patient in an amount of 6×10¹³ vg/kg.

In some embodiments of any of the above aspects of the disclosure, the AAV vector is administered to the patient in an amount of 7×10¹³ vg/kg.

In some embodiments of any of the above aspects of the disclosure, the AAV vector is administered to the patient in an amount of 8×10¹³ vg/kg.

In some embodiments of any of the above aspects of the disclosure, the AAV vector is administered to the patient in an amount of 9×10¹³ vg/kg.

In some embodiments of any of the above aspects of the disclosure, the AAV vector is administered to the patient in an amount of 1×10¹⁴ vg/kg.

In some embodiments of any of the above aspects of the disclosure, the AAV vector is administered to the patient in an amount of 1.1×10¹⁴ vg/kg.

In some embodiments of any of the above aspects of the disclosure, the AAV vector is administered to the patient in an amount of 1.2×10¹⁴ vg/kg.

In some embodiments of any of the above aspects of the disclosure, the AAV vector is administered to the patient in an amount of 1.3×10¹⁴ vg/kg.

In some embodiments of any of the above aspects of the disclosure, the AAV vector is administered to the patient in an amount of 1.4×10¹⁴ vg/kg.

In some embodiments of any of the above aspects of the disclosure, the AAV vector is administered to the patient in an amount of 1.5×10¹⁴ vg/kg.

In some embodiments of any of the above aspects of the disclosure, the AAV vector is administered to the patient in an amount of 1.6×10¹⁴ vg/kg.

In some embodiments of any of the above aspects of the disclosure, the AAV vector is administered to the patient in an amount of 1.7×10¹⁴ vg/kg.

In some embodiments of any of the above aspects of the disclosure, the AAV vector is administered to the patient in an amount of 1.8×10¹⁴ vg/kg.

In some embodiments of any of the above aspects of the disclosure, the AAV vector is administered to the patient in an amount of 1.9×10¹⁴ vg/kg.

In some embodiments of any of the above aspects of the disclosure, the AAV vector is administered to the patient in an amount of 2×10¹⁴ vg/kg.

In some embodiments of any of the above aspects of the disclosure, the AAV vector is administered to the patient in a single dose containing the specified amount. In some embodiments, the AAV vector is administered to the patient in two or more doses that, together, total the specified amount. For example, the AAV vector may be administered to the patient in from two to ten doses that, together, total the specified amount (e.g., in two, three, four, five, six, seven, eight, nine, or ten doses that, together, total the specified amount). In some embodiments, the AAV vector is administered to the patient in two, three, or four doses that, together, total the specified amount. In some embodiments, the AAV vector is administered to the patient in two doses that, together, total the specified amount.

In some embodiments, the two or more doses of the AAV vector that, together, total the specified amount are separated from one another, for example, by a year or more. In some embodiments, the two or more doses are administered to the patient within about 12 months of one another (e.g., within about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks of one another). For example, in some embodiments, the two or more doses are administered to the patient within from about one week to about 48 weeks of one another (e.g., within about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, or 48 weeks of one another). In some embodiments, the two or more doses are administered to the patient within from about two weeks to about 44 weeks of one another (e.g., within about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, or 44 weeks of one another). In some embodiments, the two or more doses are administered to the patient within from about three weeks to about 40 weeks of one another (e.g., within about 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks of one another). In some embodiments, the two or more doses are administered to the patient within from about four weeks to about 36 weeks of one another (e.g., within about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks of one another). In some embodiments, the two or more doses are administered to the patient within from about five weeks to about 32 weeks of one another (e.g., within about 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, or 32 weeks of one another). In some embodiments, the two or more doses are administered to the patient within from about six weeks to about 24 weeks of one another (e.g., within about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of one another). In some embodiments, the two or more doses are administered to the patient within from about 12 weeks to about 20 weeks of one another (e.g., within about 12 weeks, 13 weeks, 14 weeks, 15 weeks, 16 weeks, 17 weeks, 18 weeks, 19 weeks, or 20 weeks of one another). In some embodiments, the two or more doses are administered to the patient within about 13 weeks, 14 weeks, 15 weeks, 16 weeks, 17 weeks, 18 weeks, or 19 weeks of one another.

In some embodiments, the AAV vector is administered to the patient in two or more doses that each, individually, contain the specified amount. For example, the AAV vector may be administered to the patient in from two to ten doses that each, individually, contain the specified amount (e.g., in two, three, four, five, six, seven, eight, nine, or ten doses that each, individually, contain the specified amount). In some embodiments, the AAV vector is administered to the patient in two, three, or four doses that each, individually, contain the specified amount. In some embodiments, the AAV vector is administered to the patient in two doses that each, individually, contain the specified amount.

In some embodiments, the AAV vector is administered to the patient by way of intravenous, intrathecal, intracisternal, intracerebroventricular, intramuscular, intradermal, transdermal, parenteral, intranasal, subcutaneous, percutaneous, intratracheal, intraperitoneal, intraarterial, intravascular, inhalation, perfusion, lavage, and/or oral administration. For example, the AAV vector may be administered to the patient by way of intravenous, intrathecal, intracisternal, intracerebroventricular, and/or intramuscular administration. In some embodiments, the AAV vector is administered to the patient by way of intravenous and/or intrathecal administration. some embodiments, the AAV vector is administered to the patient by way of intravenous administration.

In some embodiments, the AAV is an AAV1, AAV2, AAV3, AAV4, AAV5, AAV6, AAV7, AAV8, AAV9, AAVrh74, AAVrh.8, or AAVrh.10 serotype. The AAV may be a pseudotyped AAV, such as an AAV2/8 or AAV2/9. In some embodiments, the AAV contains a recombinant capsid protein.

In some embodiments, the transgene encoding GAA is operably linked to a promoter that induces expression of the transgene in a muscle and/or neuronal cell. The promoter may be, for example, a muscle creatine kinase (MCK) promoter, desmin promoter, chicken beta actin promoter, cytomegalovirus (CMV) promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

In some embodiments, the promoter is a MCK promoter. The MCK promoter may have, for example, a nucleic acid sequence that is at least 85% identical to SEQ ID NO: 1 (e.g., a nucleic acid sequence that is 85%, 86%, 87%, 88%, 89%, 90%, 91%, 92%, 93%, 94%, 95%, 96%, 97%, 98%, 99%, or 100% identical to SEQ ID NO: 1). In some embodiments, the MCK promoter has a nucleic acid sequence that is at least 90% identical to SEQ ID NO: 1 (e.g., a nucleic acid sequence that is 90%, 91%, 92%, 93%, 94%, 95%, 96%, 97%, 98%, 99%, or 100% identical to SEQ ID NO: 1). In some embodiments, the MCK promoter has a nucleic acid sequence that is at least 95% identical to SEQ ID NO: 1 (e.g., a nucleic acid sequence that is 95%, 96%, 97%, 98%, 99%, or 100% identical to SEQ ID NO: 1). In some embodiments, the MCK promoter has a nucleic acid sequence that is at least 97% identical to SEQ ID NO: 1 (e.g., a nucleic acid sequence that is 97%, 98%, 99%, or 100% identical to SEQ ID NO: 1). In some embodiments, the MCK promoter has a nucleic acid sequence that is at least 98% identical to SEQ ID NO: 1 (e.g., a nucleic acid sequence that is 98%, 99%, or 100% identical to SEQ ID NO: 1). In some embodiments, the MCK promoter has a nucleic acid sequence that is at least 99% identical to SEQ ID NO: 1 (e.g., a nucleic acid sequence that is 99%, 99.1%, 99.2%, 99.3%, 99.4%, 99.5%, 99.6%, 99.7%, 99.8%, 99.9%, or 100% identical to SEQ ID NO: 1). In some embodiments, the MCK promoter has a nucleic acid sequence that is 100% identical to SEQ ID NO: 1.

In some embodiments, the transgene encoding GAA is operably linked to an enhancer that induces expression of the transgene in a muscle and/or neuronal cell. For example, the transgene encoding GAA may be operably linked to a CMV enhancer, a myocyte enhancer factor 2 (MEF2) enhancer, or a MyoD enhancer.

In some embodiments, the GAA has an amino acid sequence that is at least 85% identical to SEQ ID NO: 2 (e.g., an amino acid sequence that is 85%, 86%, 87%, 88%, 89%, 90%, 91%, 92%, 93%, 94%, 95%, 96%, 97%, 98%, 99%, or 100% identical to SEQ ID NO: 2). In some embodiments, the GAA has an amino acid sequence that is at least 90% identical to SEQ ID NO: 2 (e.g., an amino acid sequence that is 90%, 91%, 92%, 93%, 94%, 95%, 96%, 97%, 98%, 99%, or 100% identical to SEQ ID NO: 2). In some embodiments, the GAA has an amino acid sequence that is at least 95% identical to SEQ ID NO: 2 (e.g., an amino acid sequence that is 95%, 96%, 97%, 98%, 99%, or 100% identical to SEQ ID NO: 2). In some embodiments, the GAA has an amino acid sequence that is at least 97% identical to SEQ ID NO: 2 (e.g., an amino acid sequence that is 97%, 98%, 99%, or 100% identical to SEQ ID NO: 2). In some embodiments, the GAA has an amino acid sequence that is at least 98% identical to SEQ ID NO: 2 (e.g., an amino acid sequence that is 98%, 99%, or 100% identical to SEQ ID NO: 2). In some embodiments, the GAA has an amino acid sequence that is at least 99% identical to SEQ ID NO: 2 (e.g., an amino acid sequence that is 99%, 99.1%, 99.2%, 99.3%, 99.4%, 99.5%, 99.6%, 99.7%, 99.8%, 99.9%, or 100% identical to SEQ ID NO: 2). In some embodiments, the GAA has an amino acid sequence that is 100% identical to SEQ ID NO: 2. In some embodiments, the GAA differs from human wild-type GAA only by way of one or more conservative amino acid substitutions. In some embodiments, the GAA differs from human wild-type GAA by way of one or more non-conservative amino acid substitutions.

In some embodiments, the transgene encoding GAA has a nucleic acid sequence that is at least 85% identical to SEQ ID NO: 3 (e.g., a nucleic acid sequence that is 85%, 86%, 87%, 88%, 89%, 90%, 91%, 92%, 93%, 94%, 95%, 96%, 97%, 98%, 99%, or 100% identical to SEQ ID NO: 3). In some embodiments, the transgene encoding GAA has a nucleic acid sequence that is at least 90% identical to SEQ ID NO: 3 (e.g., a nucleic acid sequence that is 90%, 91%, 92%, 93%, 94%, 95%, 96%, 97%, 98%, 99%, or 100% identical to SEQ ID NO: 3). In some embodiments, the transgene encoding GAA has a nucleic acid sequence that is at least 95% identical to SEQ ID NO: 3 (e.g., a nucleic acid sequence that is 95%, 96%, 97%, 98%, 99%, or 100% identical to SEQ ID NO: 3). In some embodiments, the transgene encoding GAA has a nucleic acid sequence that is at least 97% identical to SEQ ID NO: 3 (e.g., a nucleic acid sequence that is 97%, 98%, 99%, or 100% identical to SEQ ID NO: 3). In some embodiments, the transgene encoding GAA has a nucleic acid sequence that is at least 98% identical to SEQ ID NO: 3 (e.g., a nucleic acid sequence that is 98%, 99%, or 100% identical to SEQ ID NO: 3). In some embodiments, the transgene encoding GAA has a nucleic acid sequence that is at least 99% identical to SEQ ID NO: 3 (e.g., a nucleic acid sequence that is 99%, 99.1%, 99.2%, 99.3%, 99.4%, 99.5%, 99.6%, 99.7%, 99.8%, 99.9%, or 100% identical to SEQ ID NO: 3). In some embodiments, the transgene encoding GAA has a nucleic acid sequence that is 100% identical to SEQ ID NO: 3.

In some embodiments, the patient has infantile-onset Pompe disease. The patient may be, for example, from about one month to about one year of age (e.g., about one month, two months, three months, four months, five months, six months, seven months, eight months, nine months, ten months, eleven months, or twelve months of age). In some embodiments, the patient is from about one month to about six months of age (e.g., about month, two months, three months, four months, five months, or six months of age).

In some embodiments, prior to administration of the AAV vector to the patient, the patient exhibits a symptom selected from feeding difficulties, failure to thrive, hypotonia, progressive weakness, respiratory distress, severe enlargement of the tongue, and thickening of the heart muscle.

In some embodiments, the patient has late-onset Pompe disease. The patient may exhibit, for example, endogenous GAA activity of from about 1% to about 40% of the endogenous GAA activity of a human of the same gender and similar body mass index that does not have Pompe disease.

In some embodiments, the patient has not previously received GAA enzyme replacement therapy. In some embodiments, the patient has previously received GAA enzyme replacement therapy.

In some embodiments, following administration of the AAV vector to the patient, the patient exhibits endogenous GAA activity of from about 50% to about 200% of the endogenous GAA activity of a human of the same gender and similar body mass index that does not have Pompe disease.

In some embodiments, following administration of the AAV vector to the patient, the patient exhibits a reduction in glycogen in skeletal muscle, cardiac muscle, and/or neuronal tissue.

In another aspect, the disclosure features a method of treating Pompe disease in a human patient in need thereof by administering to the patient an agent that increases GAA expression. In accordance with this aspect, the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector containing a transgene encoding GAA in an amount of from about 1×10¹³ vg/kg to about 5×10¹⁴ vg/kg (e.g., in an amount of from about 1×10¹³ vg/kg to about 3×10¹⁴ vg/kg), wherein the transgene encoding GAA is operably linked to a MCK promoter. For example, the agent may be administered to the patient in an amount sufficient to achieve a level of GAA activity in the patient that is equivalent to a level of GAA activity observed in the human subject of the same gender and similar body mass index upon administration to the subject of the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a method of improving muscle function in a human patient diagnosed as having Pompe disease by administering to the patient an agent that increases GAA expression. In accordance with this aspect, the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector containing a transgene encoding GAA in an amount of from about 1×10¹³ vg/kg to about 5×10¹⁴ vg/kg (e.g., in an amount of from about 1×10¹³ vg/kg to about 3×10¹⁴ vg/kg), wherein the transgene encoding GAA is operably linked to a MCK promoter. For example, the agent may be administered to the patient in an amount sufficient to achieve a level of GAA activity in the patient that is equivalent to a level of GAA activity observed in the human subject of the same gender and similar body mass index upon administration to the subject of the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a method of reducing glycogen accumulation in a human patient diagnosed as having Pompe disease by administering to the patient an agent that increases GAA expression. In accordance with this aspect, the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector containing a transgene encoding GAA in an amount of from about 1×10¹³ vg/kg to about 5×10¹⁴ vg/kg (e.g., in an amount of from about 1×10¹³ vg/kg to about 3×10¹⁴ vg/kg), wherein the transgene encoding GAA is operably linked to a MCK promoter. For example, the agent may be administered to the patient in an amount sufficient to achieve a level of GAA activity in the patient that is equivalent to a level of GAA activity observed in the human subject of the same gender and similar body mass index upon administration to the subject of the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg. In some embodiments of this aspect, administration of the agent to the patient reduces glycogen accumulation in muscle tissue (e.g., in cardiac and/or skeletal muscle tissue) and/or in neuronal tissue.

In another aspect, the disclosure features a method of improving pulmonary function in a human patient diagnosed as having Pompe disease by administering to the patient an agent that increases GAA expression. In accordance with this aspect, the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector containing a transgene encoding GAA in an amount of from about 1×10¹³ vg/kg to about 5×10¹⁴ vg/kg (e.g., in an amount of from about 1×10¹³ vg/kg to about 3×10¹⁴ vg/kg), wherein the transgene encoding GAA is operably linked to a MCK promoter. For example, the agent may be administered to the patient in an amount sufficient to achieve a level of GAA activity in the patient that is equivalent to a level of GAA activity observed in the human subject of the same gender and similar body mass index upon administration to the subject of the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a method of increasing GAA expression in a human patient diagnosed as having Pompe disease by administering to the patient an agent that increases GAA expression. In accordance with this aspect, the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector containing a transgene encoding GAA in an amount of from about 1×10¹³ vg/kg to about 5×10¹⁴ vg/kg (e.g., in an amount of from about 1×10¹³ vg/kg to about 3×10¹⁴ vg/kg), wherein the transgene encoding GAA is operably linked to a MCK promoter. For example, the agent may be administered to the patient in an amount sufficient to achieve a level of GAA activity in the patient that is equivalent to a level of GAA activity observed in the human subject of the same gender and similar body mass index upon administration to the subject of the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg. In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 3×10¹³ vg/kg to about 2×10¹⁴ vg/kg, such as an in amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 4×10¹³ vg/kg to about 2×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 5×10¹³ vg/kg to about 2×10¹⁴ vg/kg, such as an in amount of 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 6×10¹³ vg/kg to about 2×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 7×10¹³ vg/kg to about 2×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 8×10¹³ vg/kg to about 2×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 9×10¹³ vg/kg to about 2×10¹⁴ vg/kg, such as an in amount of about 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 1×10¹⁴ vg/kg to about 2×10¹⁴ vg/kg, such as an in amount of about 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 6×10¹³ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 7×10¹³ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 8×10¹³ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 9×10¹³ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.1×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.2×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.3×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.4×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.5×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.6×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.7×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.8×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.9×10¹⁴ vg/kg.

In some embodiments of any of the preceding five aspects of the disclosure, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 2×10¹⁴ vg/kg.

In some embodiments, the agent is administered to the patient in a single dose. In some embodiments, the agent is administered to the patient in two or more doses.

In some embodiments, the agent is administered to the patient by way of intravenous, intrathecal, intracisternal, intracerebroventricular, intramuscular, intradermal, transdermal, parenteral, intranasal, subcutaneous, percutaneous, intratracheal, intraperitoneal, intraarterial, intravascular, inhalation, perfusion, lavage, and/or oral administration.

In some embodiments, the agent contains (i) a nucleic acid molecule encoding GAA, (ii) one or more interfering RNA molecules that collectively increase expression of endogenous GAA, (iii) one or more nucleic acid molecules encoding the one or more interfering RNA molecules, (iv) a GAA protein, and/or (v) one or more small molecules that collectively increase expression of endogenous GAA. For example, the agent may be one that contains one or more interfering RNA molecules that comprise short interfering RNA (siRNA), short hairpin RNA (shRNA), and/or micro RNA (miRNA).

In some embodiments, the agent contains a nucleic acid molecule encoding GAA. The nucleic acid molecule encoding GAA may be provided to the patient, for example, by administering to the patient a viral vector that contains the nucleic acid molecule. The viral vector may be, for example, an AAV, an adenovirus, a parvovirus, a coronavirus, a rhabdovirus, a paramyxovirus, a picornavirus, an alphavirus, a herpes virus, a poxvirus, or a Retroviridae family virus.

In some embodiments, the nucleic acid molecule encoding GAA is provided to the patient by administering to the patient an AAV containing the nucleic acid molecule. The AAV may have, for example, a serotype selected from AAV1, AAV2, AAV3, AAV4, AAV5, AAV6, AAV7, AAV8, AAV9, AAV10, and AAVrh74. In some embodiments, the AAV is a pseudotyped AAV. In some embodiments, the pseudotyped AAV is AAV2/8. In some embodiments, the AAV is a pseudotyped AAV. In some embodiments, the pseudotyped AAV is AAV2/9. In some embodiments, the AAV contains a recombinant capsid protein.

In some embodiments, the nucleic acid molecule encoding GAA is operably linked to a promoter that induces expression of the transgene in a muscle and/or neuronal cell. The promoter may be, for example, a MCK promoter, desmin promoter, chicken beta actin promoter, CMV promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

In some embodiments, the promoter is a MCK promoter. The MCK promoter may have, for example, a nucleic acid sequence that is at least 85% identical to SEQ ID NO: 1 (e.g., a nucleic acid sequence that is 85%, 86%, 87%, 88%, 89%, 90%, 91%, 92%, 93%, 94%, 95%, 96%, 97%, 98%, 99%, or 100% identical to SEQ ID NO: 1). In some embodiments, the MCK promoter has a nucleic acid sequence that is at least 90% identical to SEQ ID NO: 1 (e.g., a nucleic acid sequence that is 90%, 91%, 92%, 93%, 94%, 95%, 96%, 97%, 98%, 99%, or 100% identical to SEQ ID NO: 1). In some embodiments, the MCK promoter has a nucleic acid sequence that is at least 95% identical to SEQ ID NO: 1 (e.g., a nucleic acid sequence that is 95%, 96%, 97%, 98%, 99%, or 100% identical to SEQ ID NO: 1). In some embodiments, the MCK promoter has a nucleic acid sequence that is at least 97% identical to SEQ ID NO: 1 (e.g., a nucleic acid sequence that is 97%, 98%, 99%, or 100% identical to SEQ ID NO: 1). In some embodiments, the MCK promoter has a nucleic acid sequence that is at least 98% identical to SEQ ID NO: 1 (e.g., a nucleic acid sequence that is 98%, 99%, or 100% identical to SEQ ID NO: 1). In some embodiments, the MCK promoter has a nucleic acid sequence that is at least 99% identical to SEQ ID NO: 1 (e.g., a nucleic acid sequence that is 99%, 99.1%, 99.2%, 99.3%, 99.4%, 99.5%, 99.6%, 99.7%, 99.8%, 99.9%, or 100% identical to SEQ ID NO: 1). In some embodiments, the MCK promoter has a nucleic acid sequence that is 100% identical to SEQ ID NO: 1.

In some embodiments, the nucleic acid molecule encoding GAA is operably linked to an enhancer that induces expression of the transgene in a muscle and/or neuronal cell. For example, the nucleic acid molecule encoding GAA may be operably linked to a CMV enhancer, a MEF2 enhancer, or a MyoD enhancer.

In some embodiments, the GAA has an amino acid sequence that is at least 85% identical to SEQ ID NO: 2 (e.g., an amino acid sequence that is 85%, 86%, 87%, 88%, 89%, 90%, 91%, 92%, 93%, 94%, 95%, 96%, 97%, 98%, 99%, or 100% identical to SEQ ID NO: 2). In some embodiments, the GAA has an amino acid sequence that is at least 90% identical to SEQ ID NO: 2 (e.g., an amino acid sequence that is 90%, 91%, 92%, 93%, 94%, 95%, 96%, 97%, 98%, 99%, or 100% identical to SEQ ID NO: 2). In some embodiments, the GAA has an amino acid sequence that is at least 95% identical to SEQ ID NO: 2 (e.g., an amino acid sequence that is 95%, 96%, 97%, 98%, 99%, or 100% identical to SEQ ID NO: 2). In some embodiments, the GAA has an amino acid sequence that is at least 97% identical to SEQ ID NO: 2 (e.g., an amino acid sequence that is 97%, 98%, 99%, or 100% identical to SEQ ID NO: 2). In some embodiments, the GAA has an amino acid sequence that is at least 98% identical to SEQ ID NO: 2 (e.g., an amino acid sequence that is 98%, 99%, or 100% identical to SEQ ID NO: 2). In some embodiments, the GAA has an amino acid sequence that is at least 99% identical to SEQ ID NO: 2 (e.g., an amino acid sequence that is 99%, 99.1%, 99.2%, 99.3%, 99.4%, 99.5%, 99.6%, 99.7%, 99.8%, 99.9%, or 100% identical to SEQ ID NO: 2). In some embodiments, the GAA has an amino acid sequence that is 100% identical to SEQ ID NO: 2. In some embodiments, the GAA differs from human wild-type GAA only by way of one or more conservative amino acid substitutions. In some embodiments, the GAA differs from human wild-type GAA by way of one or more non-conservative amino acid substitutions.

In some embodiments, the nucleic acid molecule encoding GAA has a nucleic acid sequence that is at least 85% identical to SEQ ID NO: 3 (e.g., a nucleic acid sequence that is 85%, 86%, 87%, 88%, 89%, 90%, 91%, 92%, 93%, 94%, 95%, 96%, 97%, 98%, 99%, or 100% identical to SEQ ID NO: 3). In some embodiments, the nucleic acid molecule encoding GAA has a nucleic acid sequence that is at least 90% identical to SEQ ID NO: 3 (e.g., a nucleic acid sequence that is 90%, 91%, 92%, 93%, 94%, 95%, 96%, 97%, 98%, 99%, or 100% identical to SEQ ID NO: 3). In some embodiments, the nucleic acid molecule encoding GAA has a nucleic acid sequence that is at least 95% identical to SEQ ID NO: 3 (e.g., a nucleic acid sequence that is 95%, 96%, 97%, 98%, 99%, or 100% identical to SEQ ID NO: 3). In some embodiments, the nucleic acid molecule encoding GAA has a nucleic acid sequence that is at least 97% identical to SEQ ID NO: 3 (e.g., a nucleic acid sequence that is 97%, 98%, 99%, or 100% identical to SEQ ID NO: 3). In some embodiments, the nucleic acid molecule encoding GAA has a nucleic acid sequence that is at least 98% identical to SEQ ID NO: 3 (e.g., a nucleic acid sequence that is 98%, 99%, or 100% identical to SEQ ID NO: 3). In some embodiments, the nucleic acid molecule encoding GAA has a nucleic acid sequence that is at least 99% identical to SEQ ID NO: 3 (e.g., a nucleic acid sequence that is 99%, 99.1%, 99.2%, 99.3%, 99.4%, 99.5%, 99.6%, 99.7%, 99.8%, 99.9%, or 100% identical to SEQ ID NO: 3). In some embodiments, the nucleic acid molecule encoding GAA has a nucleic acid sequence that is 100% identical to SEQ ID NO: 3.

In some embodiments, the patient has infantile-onset Pompe disease. The patient may be, for example, from about one month to about one year of age (e.g., about one month, two months, three months, four months, five months, six months, seven months, eight months, nine months, ten months, eleven months, or twelve months of age). In some embodiments, the patient is from about one month to about six months of age (e.g., about month, two months, three months, four months, five months, or six months of age).

In some embodiments, prior to administration of the AAV vector to the patient, the patient exhibits a symptom selected from feeding difficulties, failure to thrive, hypotonia, progressive weakness, respiratory distress, severe enlargement of the tongue, and thickening of the heart muscle.

In some embodiments, the patient has late-onset Pompe disease. The patient may exhibit, for example, endogenous GAA activity of from about 1% to about 40% of the endogenous GAA activity of a human of the same gender and similar body mass index that does not have Pompe disease.

In some embodiments, the patient has not previously received GAA enzyme replacement therapy. In some embodiments, the patient has previously received GAA enzyme replacement therapy.

In some embodiments, following administration of the AAV vector to the patient, the patient exhibits endogenous GAA activity of from about 50% to about 200% of the endogenous GAA activity of a human of the same gender and similar body mass index that does not have Pompe disease.

In some embodiments, following administration of the AAV vector to the patient, the patient exhibits a reduction in glycogen in skeletal muscle, cardiac muscle, and/or neuronal tissue.

In another aspect, the disclosure features a kit containing (i) an AAV vector containing a transgene encoding GAA, e.g., in an amount specified above, or (ii) an agent that increases GAA expression, e.g., in an amount specified above. The kit may further contain, for example, a package insert that instructs a user of the kit to administer the AAV vector or agent to a human patient in accordance with the method of any of the above aspects of the disclosure.

In another aspect, the disclosure features a use of an AAV vector containing a transgene encoding GAA in the manufacture of a medicament for treating Pompe disease in a human patient in need thereof, wherein the medicament contains the AAV vector in an amount of from about 1×10¹³ vg/kg to about 5×10¹⁴ vg/kg (e.g., in an amount of from about 1×10¹³ vg/kg to about 3×10¹⁴ vg/kg). For example, the medicament may contain the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a use of an AAV vector containing a transgene encoding GAA in the manufacture of a medicament for improving muscle function in a human patient diagnosed as having Pompe disease, wherein the medicament contains the AAV vector in an amount of from about 1×10¹³ vg/kg to about 5×10¹⁴ vg/kg (e.g., in an amount of from about 1×10¹³ vg/kg to about 3×10¹⁴ vg/kg). For example, the medicament may contain the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a use of an AAV vector containing a transgene encoding GAA in the manufacture of a medicament for reducing glycogen accumulation (e.g., in muscle and/or neuronal tissue) in a human patient diagnosed as having Pompe disease, wherein the medicament contains the AAV vector in an amount of from about 1×10¹³ vg/kg to about 5×10¹⁴ vg/kg (e.g., in an amount of from about 1×10¹³ vg/kg to about 3×10¹⁴ vg/kg). For example, the medicament may contain the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a use of an AAV vector containing a transgene encoding GAA in the manufacture of a medicament for improving pulmonary function in a human patient diagnosed as having Pompe disease, wherein the medicament contains the AAV vector in an amount of from about 1×10¹³ vg/kg to about 5×10¹⁴ vg/kg (e.g., in an amount of from about 1×10¹³ vg/kg to about 3×10¹⁴ vg/kg). For example, the medicament may contain the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In another aspect, the disclosure features a use of an AAV vector containing a transgene encoding GAA in the manufacture of a medicament for increasing GAA expression in a human patient diagnosed as having Pompe disease, wherein the medicament contains the AAV vector in an amount of from about 1×10¹³ vg/kg to about 5×10¹⁴ vg/kg (e.g., in an amount of from about 1×10¹³ vg/kg to about 3×10¹⁴ vg/kg). For example, the medicament may contain the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, 3×10¹⁴ vg/kg, 3.1×10¹⁴ vg/kg, 3.2×10¹⁴ vg/kg, 3.3×10¹⁴ vg/kg, 3.4×10¹⁴ vg/kg, 3.5×10¹⁴ vg/kg, 3.6×10¹⁴ vg/kg, 3.7×10¹⁴ vg/kg, 3.8×10¹⁴ vg/kg, 3.9×10¹⁴ vg/kg, 4×10¹⁴ vg/kg, 4.1×10¹⁴ vg/kg, 4.2×10¹⁴ vg/kg, 4.3×10¹⁴ vg/kg, 4.4×10¹⁴ vg/kg, 4.5×10¹⁴ vg/kg, 4.6×10¹⁴ vg/kg, 4.7×10¹⁴ vg/kg, 4.8×10¹⁴ vg/kg, 4.9×10¹⁴ vg/kg, or 5×10¹⁴ vg/kg.

In some embodiments of any of the preceding five aspects of the disclosure, the medicament contains the AAV vector in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the medicament contains the AAV vector in an amount of from about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

In some embodiments of any of the preceding five aspects of the disclosure, the medicament contains the AAV vector in an amount of from about 3×10¹³ vg/kg to about 2×10¹⁴ vg/kg, such as an in amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of from about 4×10¹³ vg/kg to about 2×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of from about 5×10¹³ vg/kg to about 2×10¹⁴ vg/kg, such as an in amount of about 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of from about 6×10¹³ vg/kg to about 2×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of from about 7×10¹³ vg/kg to about 2×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of from about 8×10¹³ vg/kg to about 2×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of from about 9×10¹³ vg/kg to about 2×10¹⁴ vg/kg, such as an in amount of about 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of from about 1×10¹⁴ vg/kg to about 2×10¹⁴ vg/kg, such as an in amount of about 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments of any of the preceding five aspects of the disclosure, the medicament contains the AAV vector in an amount of about 6×10¹³ vg/kg.

In some embodiments of any of the preceding five aspects of the disclosure, the medicament contains the AAV vector in an amount of about 7×10¹³ vg/kg.

In some embodiments of any of the preceding five aspects of the disclosure, the medicament contains the AAV vector in an amount of about 8×10¹³ vg/kg.

In some embodiments of any of the preceding five aspects of the disclosure, the medicament contains the AAV vector in an amount of about 9×10¹³ vg/kg.

In some embodiments of any of the preceding five aspects of the disclosure, the medicament contains the AAV vector in an amount of about 1×10¹⁴ vg/kg.

In some embodiments of any of the preceding five aspects of the disclosure, the medicament contains the AAV vector in an amount of about 1.1×10¹⁴ vg/kg.

In some embodiments of any of the preceding five aspects of the disclosure, the medicament contains the AAV vector in an amount of about 1.2×10¹⁴ vg/kg.

In some embodiments of any of the preceding five aspects of the disclosure, the medicament contains the AAV vector in an amount of about 1.3×10¹⁴ vg/kg.

In some embodiments of any of the preceding five aspects of the disclosure, the medicament contains the AAV vector in an amount of about 1.4×10¹⁴ vg/kg.

In some embodiments of any of the preceding five aspects of the disclosure, the medicament contains the AAV vector in an amount of about 1.5×10¹⁴ vg/kg.

In some embodiments of any of the preceding five aspects of the disclosure, the medicament contains the AAV vector in an amount of about 1.6×10¹⁴ vg/kg.

In some embodiments of any of the preceding five aspects of the disclosure, the medicament contains the AAV vector in an amount of about 1.7×10¹⁴ vg/kg.

In some embodiments of any of the preceding five aspects of the disclosure, the medicament contains the AAV vector in an amount of about 1.8×10¹⁴ vg/kg.

In some embodiments of any of the preceding five aspects of the disclosure, the medicament contains the AAV vector in an amount of about 1.9×10¹⁴ vg/kg.

In some embodiments of any of the preceding five aspects of the disclosure, the medicament contains the AAV vector in an amount of about 2×10¹⁴ vg/kg.

In some embodiments, the AAV vector is formulated for administration to the patient in a single dose containing the amount. In some embodiments, the AAV vector is formulated for administration to the patient in two or more doses that, together, total the specified amount (e.g., in two, three, four, five, six, seven, eight, nine, or ten doses that, together, total the specified amount). In some embodiments, the AAV vector is formulated for administration to the patient in two or more doses that each, individually, contain the specified amount (e.g., in two, three, four, five, six, seven, eight, nine, or ten doses that each, individually, contain the specified amount).

In some embodiments, the AAV vector is formulated for intravenous, intrathecal, intracisternal, intracerebroventricular, intramuscular, intradermal, transdermal, parenteral, intranasal, subcutaneous, percutaneous, intratracheal, intraperitoneal, intraarterial, intravascular, inhalation, perfusion, lavage, and/or oral administration to the patient. For example, the AAV vector may be formulated for intravenous, intrathecal, intracisternal, intracerebroventricular, and/or intramuscular administration to the patient. In some embodiments, the AAV vector is formulated for intravenous and/or intrathecal administration to the patient. The AAV vector may be, for example, formulated for intravenous administration to the patient.

In some embodiments, the AAV is an AAV1, AAV2, AAV3, AAV4, AAV5, AAV6, AAV7, AAV8, AAV9, AAVrh74, AAVrh.8, or AAVrh.10 serotype. In some embodiments, the AAV is a pseudotyped AAV, such as an AAV2/8 or AAV2/9. In some embodiments, the AAV contains a recombinant capsid protein.

In some embodiments, the transgene encoding GAA is operably linked to a promoter that induces expression of the transgene in a muscle and/or neuronal cell. The promoter may be, for example, a MCK promoter, desmin promoter, chicken beta actin promoter, CMV promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

In some embodiments, the promoter is a MCK promoter. The MCK promoter may have, for example, a nucleic acid sequence that is at least 85% identical to SEQ ID NO: 1 (e.g., a nucleic acid sequence that is 85%, 86%, 87%, 88%, 89%, 90%, 91%, 92%, 93%, 94%, 95%, 96%, 97%, 98%, 99%, or 100% identical to SEQ ID NO: 1). In some embodiments, the MCK promoter has a nucleic acid sequence that is at least 90% identical to SEQ ID NO: 1 (e.g., a nucleic acid sequence that is 90%, 91%, 92%, 93%, 94%, 95%, 96%, 97%, 98%, 99%, or 100% identical to SEQ ID NO: 1). In some embodiments, the MCK promoter has a nucleic acid sequence that is at least 95% identical to SEQ ID NO: 1 (e.g., a nucleic acid sequence that is 95%, 96%, 97%, 98%, 99%, or 100% identical to SEQ ID NO: 1). In some embodiments, the MCK promoter has a nucleic acid sequence that is at least 97% identical to SEQ ID NO: 1 (e.g., a nucleic acid sequence that is 97%, 98%, 99%, or 100% identical to SEQ ID NO: 1). In some embodiments, the MCK promoter has a nucleic acid sequence that is at least 98% identical to SEQ ID NO: 1 (e.g., a nucleic acid sequence that is 98%, 99%, or 100% identical to SEQ ID NO: 1). In some embodiments, the MCK promoter has a nucleic acid sequence that is at least 99% identical to SEQ ID NO: 1 (e.g., a nucleic acid sequence that is 99%, 99.1%, 99.2%, 99.3%, 99.4%, 99.5%, 99.6%, 99.7%, 99.8%, 99.9%, or 100% identical to SEQ ID NO: 1). In some embodiments, the MCK promoter has a nucleic acid sequence that is 100% identical to SEQ ID NO: 1.

In some embodiments, the transgene encoding GAA is operably linked to an enhancer that induces expression of the transgene in a muscle and/or neuronal cell. For example, the transgene encoding GAA may be operably linked to a CMV enhancer, a MEF2 enhancer, or a MyoD enhancer.

In some embodiments, the GAA has an amino acid sequence that is at least 85% identical to SEQ ID NO: 2 (e.g., an amino acid sequence that is 85%, 86%, 87%, 88%, 89%, 90%, 91%, 92%, 93%, 94%, 95%, 96%, 97%, 98%, 99%, or 100% identical to SEQ ID NO: 2). In some embodiments, the GAA has an amino acid sequence that is at least 90% identical to SEQ ID NO: 2 (e.g., an amino acid sequence that is 90%, 91%, 92%, 93%, 94%, 95%, 96%, 97%, 98%, 99%, or 100% identical to SEQ ID NO: 2). In some embodiments, the GAA has an amino acid sequence that is at least 95% identical to SEQ ID NO: 2 (e.g., an amino acid sequence that is 95%, 96%, 97%, 98%, 99%, or 100% identical to SEQ ID NO: 2). In some embodiments, the GAA has an amino acid sequence that is at least 97% identical to SEQ ID NO: 2 (e.g., an amino acid sequence that is 97%, 98%, 99%, or 100% identical to SEQ ID NO: 2). In some embodiments, the GAA has an amino acid sequence that is at least 98% identical to SEQ ID NO: 2 (e.g., an amino acid sequence that is 98%, 99%, or 100% identical to SEQ ID NO: 2). In some embodiments, the GAA has an amino acid sequence that is at least 99% identical to SEQ ID NO: 2 (e.g., an amino acid sequence that is 99%, 99.1%, 99.2%, 99.3%, 99.4%, 99.5%, 99.6%, 99.7%, 99.8%, 99.9%, or 100% identical to SEQ ID NO: 2). In some embodiments, the GAA has an amino acid sequence that is 100% identical to SEQ ID NO: 2. In some embodiments, the GAA differs from human wild-type GAA only by way of one or more conservative amino acid substitutions. In some embodiments, the GAA differs from human wild-type GAA by way of one or more non-conservative amino acid substitutions.

In some embodiments, the transgene encoding GAA has a nucleic acid sequence that is at least 85% identical to SEQ ID NO: 3 (e.g., a nucleic acid sequence that is 85%, 86%, 87%, 88%, 89%, 90%, 91%, 92%, 93%, 94%, 95%, 96%, 97%, 98%, 99%, or 100% identical to SEQ ID NO: 3). In some embodiments, the transgene encoding GAA has a nucleic acid sequence that is at least 90% identical to SEQ ID NO: 3 (e.g., a nucleic acid sequence that is 90%, 91%, 92%, 93%, 94%, 95%, 96%, 97%, 98%, 99%, or 100% identical to SEQ ID NO: 3). In some embodiments, the transgene encoding GAA has a nucleic acid sequence that is at least 95% identical to SEQ ID NO: 3 (e.g., a nucleic acid sequence that is 95%, 96%, 97%, 98%, 99%, or 100% identical to SEQ ID NO: 3). In some embodiments, the transgene encoding GAA has a nucleic acid sequence that is at least 97% identical to SEQ ID NO: 3 (e.g., a nucleic acid sequence that is 97%, 98%, 99%, or 100% identical to SEQ ID NO: 3). In some embodiments, the transgene encoding GAA has a nucleic acid sequence that is at least 98% identical to SEQ ID NO: 3 (e.g., a nucleic acid sequence that is 98%, 99%, or 100% identical to SEQ ID NO: 3). In some embodiments, the transgene encoding GAA has a nucleic acid sequence that is at least 99% identical to SEQ ID NO: 3 (e.g., a nucleic acid sequence that is 99%, 99.1%, 99.2%, 99.3%, 99.4%, 99.5%, 99.6%, 99.7%, 99.8%, 99.9%, or 100% identical to SEQ ID NO: 3). In some embodiments, the transgene encoding GAA has a nucleic acid sequence that is 100% identical to SEQ ID NO: 3.

In some embodiments, the patient has infantile-onset Pompe disease. The patient may be, for example, from about one month to about one year of age (e.g., about one month, two months, three months, four months, five months, six months, seven months, eight months, nine months, ten months, eleven months, or twelve months of age). In some embodiments, the patient is from about one month to about six months of age (e.g., about month, two months, three months, four months, five months, or six months of age).

In some embodiments, prior to administration of the AAV vector to the patient, the patient exhibits a symptom selected from feeding difficulties, failure to thrive, hypotonia, progressive weakness, respiratory distress, severe enlargement of the tongue, and thickening of the heart muscle.

In some embodiments, the patient has late-onset Pompe disease. The patient may exhibit, for example, endogenous GAA activity of from about 1% to about 40% of the endogenous GAA activity of a human of the same gender and similar body mass index that does not have Pompe disease.

In some embodiments, the patient has not previously received GAA enzyme replacement therapy. In some embodiments, the patient has previously received GAA enzyme replacement therapy.

In some embodiments, following administration of the AAV vector to the patient, the patient exhibits endogenous GAA activity of from about 50% to about 200% of the endogenous GAA activity of a human of the same gender and similar body mass index that does not have Pompe disease.

In some embodiments, following administration of the AAV vector to the patient, the patient exhibits a reduction in glycogen in skeletal muscle, cardiac muscle, and/or neuronal tissue.

### BRIEF DESCRIPTION OF THE DRAWINGS

FIGS. 1A and 1B are graphs showing changes in acid alpha-glucosidase (GAA) protein expression and enzymatic activity levels, respectively, in muscles of mice treated with an AAV2/8 vector containing an GAA transgene operably linked to a muscle creatine kinase (MCK) promoter as described in Example 1, below.

FIGS. 2A, 2B, and 2C are graphs and representative images showing changes in glycogen content in muscles of mice treated with an AAV2/8 vector containing a GAA transgene operably linked to a MCK promoter as described in Example 1, below.

FIG. 3 is a graph showing changes in motoric function over time, as measured with consecutive Grip-strength Tests, in mice treated with an AAV2/8 vector containing a GAA transgene operably linked to a MCK promoter as described in Example 1, below.

FIGS. 4A and 4B are graphs showing changes in alanine aminotransferase (ALT) activity in non-human primates treated with an AAV2/8 vector containing a GAA transgene operably linked to a MCK promoter as described in Example 2, below.

FIGS. 5A and 5B are graphs showing changes in aspartate aminotransferase (AST) activity in non-human primates treated with an AAV2/8 vector containing a GAA transgene operably linked to a MCK promoter as described in Example 2, below.

FIGS. 6A, 6B, and 6C are graphs showing changes in troponin-I expression in non-human primates (male, female, and sex-combined data, respectively) treated with an AAV2/8 vector containing a human GAA transgene operably linked to a MCK promoter as described in Example 2, below.

FIG. 7 is a graph showing changes in troponin-I expression in non-human primates treated with an AAV2/8 vector containing a cynomolgus GAA transgene operably linked to a MCK promoter as described in Example 2, below.

FIGS. 8A and 8B are graphs showing changes in brain natriuretic peptide (BNP) expression in non-human primates treated with an AAV2/8 vector containing a GAA transgene operably linked to a MCK promoter as described in Example 2, below.

FIGS. 9A and 9B are graphs showing changes in human or cynomolgus GAA protein expression, in muscles of non-human primates treated with an AAV2/8 vector containing a human GAA or cynomolgus GAA transgene, respectively, operably linked to a MCK promoter as described in Example 2, below.

FIGS. 10A and 10B are graphs showing changes in human or cynomolgus GAA protein expression in serum of non-human primates treated with an AAV2/8 vector containing a human GAA or cynomolgus GAA transgene, respectively, operably linked to a MCK promoter as described in Example 2, below.

### DEFINITIONS

As used herein, the term “about” refers to a value that is within 5% above or below the value being described. For example, “about 1×10¹³ vg/kg” as used in the context of a viral vector described herein includes quantities that are within 5% above or below 1×10¹³ vg/kg. Additionally, when used in the context of a list of numerical quantities, it is to be understood that the term “about,” when preceding a list of numerical quantities, applies to each individual quantity recited in the list. For example, “about 1×10¹³ vg/kg, 2×10¹³ vg/kg, or 3×10¹³ vg/kg” is to be construed as equivalent to individually reciting “about 1×10¹³ vg/kg,” “about 2×10¹³ vg/kg,” and “about 3×10¹³ vg/kg.”

As used herein in the context of a protein of interest, such as acid alpha-glucosidase (GAA), the term “activity” refers to the biological functionality that is associated with a wild-type form of the protein. For example, in the context of an enzyme, the term “activity” refers to the ability of the protein to effectuate substrate turnover in a manner that yields the product of a corresponding chemical reaction. Activity levels of enzymes, such as GAA, can be detected and quantitated, for example, using substrate turnover assays known in the art.

As used herein, the terms “administering,” “administration,” and the like refer to directly giving a patient a therapeutic agent (e.g., a viral vector) by any effective route. Exemplary routes of administration are described herein and include systemic administration routes, such as intravenous injection, as well as routes of administration directly to the central nervous system of the patient, such as by way of intrathecal injection or intracerebroventricular injection, among others.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15 (20): 8125-8148, incorporated herein by reference in its entirety. Multiple stop codons can be incorporated.

As used herein, the terms “conservative mutation,” “conservative substitution,” “conservative amino acid substitution,” and the like refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally occurring amino acids

| | | | Side- | Electrostatic | |
|---|---|---|---|---|---|
| | 3 Letter | 1 Letter | chain | character at | Steric |
| Amino Acid | Code | Code | Polarity | physiological pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “dose” refers to the quantity of a therapeutic agent, such as a viral vector described herein, that is administered to a subject at a particular instant for the treatment of a disorder or condition, such as to treat or ameliorate one or more symptoms of a glycogen storage disorder described herein (e.g., Pompe disease). A therapeutic agent as described herein may be administered in a single dose or in multiple doses over the course of a treatment period, as defined herein. In each case, the therapeutic agent may be administered using one or more unit dosage forms of the therapeutic agent, a term that refers to a one or more discrete compositions containing a therapeutic agent that collectively constitute a single dose of the agent. For instance, a single dose of 1×10¹³ vector genomes (vg) of a viral vector may be administered using, e.g., two 0.5×10¹³ vg unit dosage forms of the viral vector.

As used herein, the terms “effective amount,” “therapeutically effective amount,” and the like, when used in reference to a therapeutic composition, such as a vector construct described herein, refer to a quantity sufficient to, when administered to the subject, including a mammal, for example a human, effect beneficial or desired results, such as clinical results. For example, in the context of treating glycogen storage disorders, such as Pompe disease, these terms refer to an amount of the composition sufficient to achieve a treatment response as compared to the response obtained without administration of the composition of interest. An “effective amount,” “therapeutically effective amount,” or the like, of a composition, such as a vector construct of the present disclosure, also include an amount that results in a beneficial or desired result in a subject as compared to a control.

As used herein, the term “enzyme replacement therapy” refers to the administration to a subject (e.g., a mammalian subject, such as a human) suffering from a genetic loss-of-function disease of the protein that is naturally defective or deficient in the subject. For example, in the context of a subject having Pompe disease, enzyme replacement therapy refers to administration of GAA protein to such a subject. Typically, enzyme replacement therapy involves administration of the therapeutic protein to the subject chronically, over the course of multiple doses throughout the subject's life.

As used herein, the terms “express” and “expression” in the context of a gene refer to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic compound to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, a therapeutic agent is considered to be “provided” to a patient if the patient is directly administered the therapeutic agent or if the patient is administered a substance that is processed or metabolized in vivo so as to yield the therapeutic agent endogenously. For example, a patient, such as a patient having a glycogen storage disorder described herein, may be provided a nucleic acid molecule encoding a therapeutic protein (e.g., GAA) by direct administration of the nucleic acid molecule or by administration of a substance (e.g., viral vector or cell) that is processed in vivo so as to yield the desired nucleic acid molecule.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, or cells) isolated from a subject. The subject may be, for example, a patient suffering from a disease described herein, such as a lysosomal storage disorder (e.g., Pompe disease).

As used herein, the terms “subject” and “patient” refer to an organism that receives treatment for a particular disease or condition as described herein (such as a lysosomal storage disorder, e.g., Pompe disease). Examples of subjects and patients include mammals, such as humans, receiving treatment for a disease or condition described herein.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Goeddel, Gene Expression Technology: Methods in Enzymology 185 (Academic Press, San Diego, Calif., 1990).

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “treat” or “treatment” refer to therapeutic treatment, in which the object is to prevent or slow down (lessen) an undesired physiological change or disorder, such as the progression of a lysosomal storage disorder, such as Pompe disease, among others. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. In the context of lysosomal storage disorders, such as Pompe disease, treatment of a patient may manifest in one or more detectable changes, such as an increase in the concentration of GAA protein or nucleic acids (e.g., DNA or RNA, such as mRNA) encoding GAA, or an increase in GAA activity (e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or more. The concentration of GAA protein may be determined using protein detection assays known in the art, including ELISA assays described herein. The concentration of GAA-encoding nucleic acids may be determined using nucleic acid detection assays (e.g., RNA Seq assays) described herein. Additionally, treatment of a patient suffering from a lysosomal storage disorder, such as Pompe disease, may manifest in improvements in a patient's muscle function (e.g., cardiac or skeletal muscle function) as well as improvements in muscle coordination.

As used herein, the term “vector” refers to a nucleic acid, e.g., DNA or RNA, that may function as a vehicle for the delivery of a gene of interest into a cell (e.g., a mammalian cell, such as a human cell), such as for purposes of replication and/or expression. Exemplary vectors useful in conjunction with the compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein in the context of a therapeutic protein, such as GAA, the use of the protein name refers to the gene encoding the protein or the corresponding protein product, depending upon the context, as will be appreciated by one of skill in the art. The term “GAA” includes wild-type forms of the GAA gene or protein, as well as variants (e.g., splice variants, truncations, concatemers, and fusion constructs, among others) of wild-type GAA proteins that retain therapeutic activity of the wild-type GAA protein, as well as nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to an amino acid sequence of a wild-type GAA protein, such as SEQ ID NO: 2, below:

(SEQ ID NO: 2)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLST
SLPSQYITGLAEHLSPLMLSTSWTRITLWNRDLAPTPGANLYGSHPFYLA
LEDGGSAHGVFLLNSNAMDVVLQPSPALSWRSTGGILDVYIFLGPEPKSV
VQQYLDVVGYPFMPPYWGLGFHLCRWGYSSTAITRQVVENMTRAHFPLDV
QWNDLDYMDSRRDFTFNKDGFRDFPAMVQELHQGGRRYMMIVDPAISSSG
PAGSYRPYDEGLRRGVFITNETGQPLIGKVWPGSTAFPDFTNPTALAWWE
DMVAEFHDQVPFDGMWIDMNEPSNFIRGSEDGCPNNELENPPYVPGVVGG
TLQAATICASSHQFLSTHYNLHNLYGLTEAIASHRALVKARGTRPFVISR
STFAGHGRYAGHWTGDVWSSWEQLASSVPEILQFNLLGVPLVGADVCGFL
GNTSEELCVRWTQLGAFYPFMRNHNSLLSLPQEPYSFSEPAQQAMRKALT
LRYALLPHLYTLFHQAHVAGETVARPLFLEFPKDSSTWTVDHQLLWGEAL
LITPVLQAGKAEVTGYFPLGTWYDLQTVPVEALGSLPPPPAAPREPAIHS
EGQWVTLPAPLDTINVHLRAGYIIPLQGPGLTTTESRQQPMALAVALTKG
GEARGELFWDDGESLEVLERGAYTQVIFLARNNTIVNELVRVTSEGAGLQ
LQKVTVLGVATAPQQVLSNGVPVSNFTYSPDTKVLDICVSLLMGEQFLVS
WC

Similarly, as used herein in the context of a transcription regulatory element, the term “MCK promoter” refers to a wild-type MCK promoter, such as a wild-type human or murine MCK promoter, as well as variants (e.g., variants containing insertions, deletions, and/or substitutions of one or more nucleic acid residues) to the extent that the promoter retains the ability to induce expression of an operably linked gene in a muscle and/or neuronal cell. An exemplary MCK promoter that may be used in conjunction with the compositions and methods of the disclosure is shown in SEQ ID NO: 1, below:

(SEQ ID NO: 1)
CCACTACGGGTCTAGGCTGCCCATGTAAGGAGGCAAGGCCTGGGGACACC
CGAGATGCCTGGTTATAATTAACCCAGACATGTGGCTGCCCCCCCCCCCC
CAACACCTGCTGCCTGAGCCTCACCCCCACCCCGGTGCCTGGGTCTTAGG
CTCTGTACACCATGGAGGAGAAGCTCGCTCTAAAAATAACCCTGTCCCTG
GTGGATCCCCTGCATGCCCAATCAAGGCTGTGGGGGACTGAGGGCAGGCT
GTAACAGGCTTGGGGGCCAGGGCTTATACGTGCCTGGGACTCCCAAAGTA
TTACTGTTCCATGTTCCCGGCGAAGGGCCAGCTGTCCCCCGCCAGCTAGA
CTCAGCACTTAGTTTAGGAACCAGTGAGCAAGTCAGCCCTTGGGGCAGCC
CATACAAGGCCATGGGGCTGGGCAAGCTGCACGCCTGGGTCCGGGGTGGG
CACGGTGCCCGGGCAACGAGCTGAAAGCTCATCTGCTCTCAGGGGCCCCT
CCCTGGGGACAGCCCCTCCTGGCTAGTCACACCCTGTAGGCTCCTCTATA
TAACCCAGGGGCACAGGGGCTGCCCCCGGGTCAC

### DETAILED DESCRIPTION

The present disclosure provides compositions and methods that can be used for treating glycogen storage disorders, particularly, type II glycogen storage disorder, also known as Pompe disease. In accordance with the compositions and methods described herein, a patient (e.g., a human patient) having Pompe disease may be administered a viral vector, such as an adeno-associated viral (AAV) vector, that contains a transgene encoding acid alpha-glucosidase (GAA). The AAV vector may be, for example, a pseudotyped AAV vector, such as an AAV vector containing AAV2 inverted terminal repeats packaged within capsid proteins from AAV8 (AAV2/8) or AAV9 (AAV2/9). In some embodiments, the transgene is operably linked to a transcription regulatory element, such as a promoter that induces gene expression in a muscle cell and/or a neuronal cell. Exemplary promoters that may be used in conjunction with the compositions and methods of the disclosure are a muscle creatine kinase promoter, desmin promoter, and CMV promoter, among others.

The present disclosure is based, in part, on the discovery of that particular doses of AAV vectors containing a GAA transgene are capable of achieving a therapeutic increase in GAA expression and activity in patients suffering from Pompe disease while suppressing toxic side effects. Particularly, doses of AAV vectors containing a transgene encoding GAA ranging from about 1×10¹³ vg/kg to about 3×10¹⁴ vg/kg (e.g., from about 3×10¹³ vg/kg to about 2×10¹⁴ vg/kg, such as a dose of about 4×10¹³ vg/kg, 5×10¹³ vg/kg, 6×10¹³ vg/kg, 7×10¹³ vg/kg, 8×10¹³ vg/kg, 9×10¹³ vg/kg, or 1×10¹⁴ vg/kg) can engender a beneficial increase in GAA expression and activity in a patient having Pompe disease while avoiding toxic side effects that can be associated with overexpression of GAA or administration of excessive quantities of viral vector. Using the compositions and methods of the disclosure, an AAV vector may be administered to the patient in an amount that is sufficient to enhance the patient's expression of GAA and reduce cellular accumulation of glycogen in the patient's neuronal and muscle tissue, without inducing toxic side effects.

In some embodiments, the AAV vector is administered to the patient in an amount of from about 1×10¹³ vector genomes (vg) per kg of body weight of the subject (vg/kg) to about 3×10¹⁴ vg/kg (e.g., in an amount of from about 3×10¹³ vg/kg to about 2×10¹⁴ vg/kg, such as in an amount of from about 4×10¹³ vg/kg to about 1×10¹⁴ vg/kg, such as in an amount of about 4×10¹³ vg/kg, 5×10¹³ vg/kg, 6×10¹³ vg/kg, 7×10¹³ vg/kg, 8×10¹³ vg/kg, 9×10¹³ vg/kg, or 1×10¹⁴ vg/kg).

The sections that follow provide a description of therapeutic agents and dosing regimens that result in the beneficial properties described above. The following sections also describe various transgenes, transcription regulatory elements, and transduction agents that may be used in conjunction with the compositions and methods of the disclosure.

### Methods of Treatment

### Pompe Disease

Pompe disease (also known as glycogen storage disease type II, or GSD II) is caused by deficiency of the lysosomal enzyme GAA. The disease is an inborn error of metabolism in which a GAA deficiency ultimately results in glycogen accumulation in all tissues, especially striated muscle cells. In addition, the effect of glycogen accumulation within the central nervous system and its effect on skeletal muscle function have been documented.

Three clinical forms of this disorder are known: infantile, juvenile, and adult. Infantile Pompe disease has its onset shortly after birth and presents with progressive muscular weakness and cardiac failure. Infantile forms of Pompe are also characterized by a rapid development of cardiomyopathy, and patients often display myopathy and neuropathy leading to death typically in the first year of life. Symptoms in adult and juvenile patients occur later in life, and skeletal muscles and neurons are primarily involved. Patients exhibiting this form of Pompe disease eventually die due to respiratory insufficiency. Patients may exceptionally survive for more than six decades. There is a correlation between the severity of the disease and the residual acid α-glucosidase activity, the activity being 10-20% of normal in late onset and less than 2% in early onset forms of the disease.

### Human Acid Alpha-Glucosidase

The amino acid sequence of an exemplary wild-type GAA is set forth in SEQ ID NO: 2, below:

(SEQ ID NO: 2)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLST
SLPSQYITGLAEHLSPLMLSTSWTRITLWNRDLAPTPGANLYGSHPFYLA
LEDGGSAHGVFLLNSNAMDVVLQPSPALSWRSTGGILDVYIFLGPEPKSV
VQQYLDVVGYPFMPPYWGLGFHLCRWGYSSTAITRQVVENMTRAHFPLDV
QWNDLDYMDSRRDFTFNKDGFRDFPAMVQELHQGGRRYMMIVDPAISSSG
PAGSYRPYDEGLRRGVFITNETGQPLIGKVWPGSTAFPDFTNPTALAWWE
DMVAEFHDQVPFDGMWIDMNEPSNFIRGSEDGCPNNELENPPYVPGVVGG
TLQAATICASSHQFLSTHYNLHNLYGLTEAIASHRALVKARGTRPFVISR
STFAGHGRYAGHWTGDVWSSWEQLASSVPEILQFNLLGVPLVGADVCGFL
GNTSEELCVRWTQLGAFYPFMRNHNSLLSLPQEPYSFSEPAQQAMRKALT
LRYALLPHLYTLFHQAHVAGETVARPLFLEFPKDSSTWTVDHQLLWGEAL
LITPVLQAGKAEVTGYFPLGTWYDLQTVPVEALGSLPPPPAAPREPAIHS
EGQWVTLPAPLDTINVHLRAGYIIPLQGPGLTTTESRQQPMALAVALTKG
GEARGELFWDDGESLEVLERGAYTQVIFLARNNTIVNELVRVTSEGAGLQ
LQKVTVLGVATAPQQVLSNGVPVSNFTYSPDTKVLDICVSLLMGEQFLVS
WC

Exemplary genes encoding a GAA polypeptide that may be used in conjunction with the compositions and methods described herein include genes encoding the wild-type GAA protein set forth in SEQ ID NO: 2, as well as functional GAA enzymes that are at least 85% identical (e.g., 85%, 86%, 87%, 88%, 89%, 90%, 91%, 92%, 93%, 94%, 95%, 96%, 97%, 98%, 99%, 99.9%, or 100% identical) to the amino acid sequence of SEQ ID NO: 2. Genes encoding a GAA polypeptide that may be used in conjunction with the compositions and methods described herein further include those that have one or more amino acid substitutions, such as those that have one or more conservative amino acid substitutions, with respect to the amino acid sequence set forth in SEQ ID NO: 2. For instance, GAA polypeptides that may be used in conjunction with the compositions and methods described herein include those that have 1, 2, 3, 4, 5, 6, 7, 8, 9, 10, 11, 12, 13, 14, 15, 16, 17, 18, 19, 20, 25, or more, conservative amino acid substitutions with respect to the amino acid sequence of SEQ ID NO: 2.

For example, in some embodiments of the disclosure, the gene encoding a GAA polypeptide has a nucleic acid sequence that is at least 85% identical (e.g., 85%, 86%, 87%, 88%, 89%, 90%, 91%, 92%, 93%, 94%, 95%, 96%, 97%, 98%, 99%, 99.9%, or 100% identical) to the nucleic acid sequence of SEQ ID NO: 3. The nucleic acid sequence of SEQ ID NO: 3 encodes the GAA polypeptide having the amino acid sequence of SEQ ID NO: 2, above. The nucleic acid sequence of SEQ ID NO: 3 is as follows:

(SEQ ID NO: 3)
ATGGGGGTGAGGCACCCCCCCTGCAGCCACAGGCTGCTGGCTGTGTGTGC
CCTGGTCAGCCTGGCCACTGCTGCCCTGCTGGGCCACATCCTGCTGCATG
ACTTCCTGCTGGTGCCCAGAGAGCTGTCTGGCAGCAGCCCTGTGCTGGAG
GAAACCCACCCTGCCCACCAGCAGGGGGCCAGCAGGCCTGGCCCCAGGGA
TGCCCAGGCCCACCCTGGCAGGCCCAGGGCTGTGCCCACCCAGTGTGATG
TGCCCCCCAACAGCAGGTTTGACTGTGCCCCTGACAAGGCCATCACCCAG
GAGCAGTGTGAGGCCAGGGGCTGCTGCTACATCCCTGCCAAGCAGGGCCT
GCAGGGGGCCCAGATGGGCCAGCCCTGGTGCTTCTTCCCCCCCtcaTACC
CCTCCTACAAGCTGGAGAACCTGAGCAGCTCTGAGATGGGCTACACTGCC
ACCCTGACCAGGACCACCCCgACgTTCTTCCCCAAgGACATCCTGACCCT
GAGGCTGGATGTGATGATGGAGACTGAGAACAGGCTGCACTTCACCATCA
AGGACCCTGCCAACAGGAGATATGAGGTGCCCCTGGAAACCCCCCATGTG
CACAGCAGGGCCCCCAGCCCCCTGTACTCTGTGGAGTTCTCTGAGGAGCC
CTTTGGGGTGATTGTGAGGAGGCAGCTGGATGGCAGGGTGCTGCTGAACA
CCACTGTGGCCCCCCTGTTCTTTGCTGACCAGTTCCTGCAGCTGAGCACC
AGCCTGCCCAGCCAGTACATCACTGGCCTGGCTGAGCACCTGAGCCCCCT
GATGCTGAGCACCAGCTGGACCAGGATCACCCTGTGGAACAGGGACCTGG
CCCCCACCCCTGGGGCCAACCTGTATGGCAGCCACCCCTTCTACCTGGCC
CTGGAGGATGGGGGCTCTGCCCATGGGGTGTTCCTGCTGAACAGCAATGC
CATGGATGTGGTGCTGCAGCCCAGCCCTGCCCTGAGCTGGAGGAGCACTG
GGGGCATCCTGGATGTGTACATCTTCCTGGGCCCTGAGCCCAAGTCTGTG
GTGCAGCAGTACCTGGATGTGGTGGGCTACCCCTTCATGCCCCCCTACTG
GGGCCTGGGCTTCCACCTGTGCAGATGGGGCTACAGCAGCACTGCCATCA
CCAGGCAGGTGGTGGAGAACATGACCAGGGCCCACTTCCCCCTGGATGTG
CAGTGGAATGACCTGGACTACATGGACAGCAGGAGGGACTTCACCTTCAA
CAAGGATGGCTTCAGGGACTTCCCTGCCATGGTGCAGGAGCTGCACCAGG
GGGGCAGGAGATACATGATGATTGTGGACCCTGCCATCAGCAGCTCTGGC
CCTGCTGGCAGCTACAGGCCCTATGATGAGGGCCTGAGGAGGGGGGTGTT
CATCACCAATGAGACTGGCCAGCCCCTGATTGGCAAGGTCTGGCCTGGCA
GCACTGCCTTCCCTGACTTCACCAACCCCACTGCCCTGGCCTGGTGGGAG
GACATGGTGGCTGAGTTCCATGACCAGGTGCCCTTTGATGGCATGTGGAT
TGACATGAATGAGCCCAGCAACTTCATCAGGGGCTCTGAGGATGGCTGCC
CCAACAATGAGCTGGAGAACCCCCCCTATGTGCCTGGGGTGGTGGGGGGC
ACCCTGCAGGCTGCCACCATCTGTGCCAGCAGCCACCAGTTCCTGAGCAC
CCACTACAACCTGCACAACCTGTATGGCCTGACTGAGGCCATTGCCAGCC
ACAGGGCCCTGGTGAAGGCCAGGGGCACCAGGCCCTTTGTGATCAGCAGG
AGCACCTTTGCTGGCCATGGCAGATATGCTGGCCACTGGACTGGGGATGT
GTGGAGCAGCTGGGAGCAGCTGGCCAGCTCTGTGCCTGAGATCCTGCAGT
TCAACCTGCTGGGGGTGCCCCTGGTGGGGGCTGATGTGTGTGGCTTCCTG
GGCAACACCTCTGAGGAGCTGTGTGTGAGGTGGACCCAGCTGGGGGCCTT
CTACCCCTTCATGAGGAACCACAACAGCCTGCTGAGCCTGCCCCAGGAGC
CCTACAGCTTCTCTGAGCCTGCCCAGCAGGCCATGAGGAAGGCCCTGACC
CTGAGATATGCCCTGCTGCCCCACCTGTACACCCTGTTCCACCAGGCCCA
TGTGGCTGGGGAGACTGTGGCCAGGCCCCTGTTCCTGGAGTTCCCCAAGG
ACAGCAGCACCTGGACTGTGGACCACCAGCTGCTGTGGGGGGAGGCCCTG
CTGATCACCCCTGTGCTGCAAGCTGGCAAGGCTGAGGTGACTGGCTACTT
CCCCCTGGGCACTTGGTATGACCTGCAGACTGTGCCTGTGGAGGCCCTGG
GCAGCCTGCCCCCCCCCCCTGCTGCCCCacggGAGCCTGCCATCCACTCT
GAGGGCCAGTGGGTGACCCTGCCTGCCCCCCTGGACACCATCAATGTGCA
CCTGAGGGCTGGCTACATCATCCCCCTGCAAGGCCCTGGCCTGACCACCA
CTGAGAGCAGGCAGCAGCCCATGGCCCTGGCTGTGGCCCTGACCAAGGGG
GGGGAGGCCAGGGGGGAGCTGTTCTGGGATGATGGGGAGAGCCTGGAGGT
GCTGGAGAGGGGGGCCTACACCCAGGTGATCTTCCTGGCCAGGAACAACA
CCATTGTGAATGAGCTGGTGAGGGTGACCTCTGAGGGGGCTGGCCTGCAG
CTGCAGAAGGTCACTGTGCTGGGGGTGGCCACTGCCCCCCAGCAGGTGCT
GAGCAATGGGGTGCCTGTGAGCAACTTCACCTACAGCCCTGACACCAAGG
TGCTGGACATCTGTGTGAGCCTGCTGATGGGGGAGCAGTTCCTGGTCAGC
TGGTGCTGA

The transcription regulatory elements described herein can be operably linked to a transgene, such as GAA, that is deficient in lysosomal storage disease patients, such as those suffering from Pompe disease. Constructs containing a lysosomal enzyme under the transcriptional control of a regulatory element described herein can be incorporated into a vector (or other transfection agent described herein) and administered to a patient so as to treat a lysosomal storage disorder. Advantageously, the therapeutic agents (e.g., viral vectors) containing a transgene described herein may promote transcription of the gene encoding the deficient lysosomal enzyme (e.g., GAA) in those cells that are affected by the disease, such as muscle cells and cells of the central nervous system. Further, the therapeutic agents described herein impart the additional benefit of avoiding toxicity that may be associated with overexpression of GAA or administration of high quantities of a viral vector encoding the same. The advantageous properties of the viral vectors and dosing regimens described herein are reported in further detail in Example 1, below.

### Dosing Regimens

### Dosing Regimens Involving AAV-GAA Vectors

Using the compositions and methods of the disclosure, a patient having a glycogen storage disorder (e.g., Pompe disease) may be administered an AAV vector containing a transgene encoding GAA in an amount of from about 1×10¹³ vg/kg to about 5×10¹⁴ vg/kg (e.g., in an amount of from about 1×10¹³ vg/kg to about 3×10¹⁴ vg/kg). For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg. Administration of the vector to the patient in such a quantity can achieve the beneficial effect of augmenting GAA expression in the patient, e.g., to within 50% or 200% of wild-type levels, without inducing toxic side effects.

For example, in some embodiments, the AAV vector is administered to the patient in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the AAV vector is administered to the patient in an amount of from about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

In some embodiments, the AAV vector is administered to the patient in an amount of from about 3×10¹³ vg/kg to about 2×10¹⁴ vg/kg, such as an in amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 4×10¹³ vg/kg to about 2×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 5×10¹³ vg/kg to about 2×10¹⁴ vg/kg, such as an in amount of about 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 6×10¹³ vg/kg to about 2×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 7×10¹³ vg/kg to about 2×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 8×10¹³ vg/kg to about 2×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 9×10¹³ vg/kg to about 2×10¹⁴ vg/kg, such as an in amount of 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 1×10¹⁴ vg/kg to about 2×10¹⁴ vg/kg, such as an in amount 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of 6×10¹³ vg/kg. In some embodiments, the AAV vector is administered to the patient in an amount of 7×10¹³ vg/kg. In some embodiments, the AAV vector is administered to the patient in an amount of 8×10¹³ vg/kg. In some embodiments, the AAV vector is administered to the patient in an amount of 9×10¹³ vg/kg. In some embodiments, the AAV vector is administered to the patient in an amount of 1×10¹⁴ vg/kg. In some embodiments, the AAV vector is administered to the patient in an amount of 1.1×10¹⁴ vg/kg. In some embodiments, the AAV vector is administered to the patient in an amount of 1.2×10¹⁴ vg/kg. In some embodiments, the AAV vector is administered to the patient in an amount of 1.3×10¹⁴ vg/kg. In some embodiments, the AAV vector is administered to the patient in an amount of 1.4×10¹⁴ vg/kg. In some embodiments, the AAV vector is administered to the patient in an amount of 1.5×10¹⁴ vg/kg. In some embodiments, the AAV vector is administered to the patient in an amount of 1.6×10¹⁴ vg/kg. In some embodiments, the AAV vector is administered to the patient in an amount of 1.7×10¹⁴ vg/kg. In some embodiments, the AAV vector is administered to the patient in an amount of 1.8×10¹⁴ vg/kg. In some embodiments, the AAV vector is administered to the patient in an amount of 1.9×10¹⁴ vg/kg. In some embodiments, the AAV vector is administered to the patient in an amount of 2×10¹⁴ vg/kg.

AAV vectors described herein may be administered to the patient in a single dose containing the specified amount. In some embodiments, the AAV vector is administered to the patient in two or more doses that, together, total the specified amount. For example, the AAV vector may be administered to the patient in from two to ten doses that, together, total the specified amount (e.g., in two, three, four, five, six, seven, eight, nine, or ten doses that, together, total the specified amount). In some embodiments, the AAV vector is administered to the patient in two, three, or four doses that, together, total the specified amount. In some embodiments, the AAV vector is administered to the patient in two doses that, together, total the specified amount.

In some embodiments, the two or more doses of the AAV vector that, together, total the specified amount are separated from one another, for example, by a year or more. In some embodiments, the two or more doses are administered to the patient within about 12 months of one another (e.g., within about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks of one another). For example, in some embodiments, the two or more doses are administered to the patient within from about one week to about 48 weeks of one another (e.g., within about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, or 48 weeks of one another). In some embodiments, the two or more doses are administered to the patient within from about two weeks to about 44 weeks of one another (e.g., within about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, or 44 weeks of one another). In some embodiments, the two or more doses are administered to the patient within from about three weeks to about 40 weeks of one another (e.g., within about 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks of one another). In some embodiments, the two or more doses are administered to the patient within from about four weeks to about 36 weeks of one another (e.g., within about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks of one another). In some embodiments, the two or more doses are administered to the patient within from about five weeks to about 32 weeks of one another (e.g., within about 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, or 32 weeks of one another). In some embodiments, the two or more doses are administered to the patient within from about six weeks to about 24 weeks of one another (e.g., within about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of one another). In some embodiments, the two or more doses are administered to the patient within from about 12 weeks to about 20 weeks of one another (e.g., within about 12 weeks, 13 weeks, 14 weeks, 15 weeks, 16 weeks, 17 weeks, 18 weeks, 19 weeks, or 20 weeks of one another). In some embodiments, the two or more doses are administered to the patient within about 13 weeks, 14 weeks, 15 weeks, 16 weeks, 17 weeks, 18 weeks, or 19 weeks of one another.

In some embodiments, the AAV vector is administered to the patient in two or more doses that each, individually, contain the specified amount. For example, the AAV vector may be administered to the patient in from two to ten doses that each, individually, contain the specified amount (e.g., in two, three, four, five, six, seven, eight, nine, or ten doses that each, individually, contain the specified amount). In some embodiments, the AAV vector is administered to the patient in two, three, or four doses that each, individually, contain the specified amount. In some embodiments, the AAV vector is administered to the patient in two doses that each, individually, contain the specified amount.

Dosing Regimens Involving Agents that Achieve GAA Expression Levels Similar to AAV2/8-MCK-GAA

Using the compositions and methods of the disclosure, a human patient having a glycogen storage disorder (e.g., Pompe disease) may be administered an agent that promotes expression of GAA in an amount sufficient to stimulate the GAA expression observed when a human subject of the same gender and similar body mass index is administered an AAV2/8 vector containing a GAA transgene under the control of a MCK promoter. For example, the agent may be administered to the patient in an amount sufficient to achieve a level of GAA activity in the patient that is equivalent to a level of GAA activity observed in the human subject of the same gender and similar body mass index upon administration to the subject of the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg.

For example, in some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 3×10¹³ vg/kg to about 2×10¹⁴ vg/kg, such as an in amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 4×10¹³ vg/kg to about 2×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 5×10¹³ vg/kg to about 2×10¹⁴ vg/kg, such as an in amount of 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 6×10¹³ vg/kg to about 2×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 7×10¹³ vg/kg to about 2×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 8×10¹³ vg/kg to about 2×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 9×10¹³ vg/kg to about 2×10¹⁴ vg/kg, such as an in amount of about 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 1×10¹⁴ vg/kg to about 2×10¹⁴ vg/kg, such as an in amount of about 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 6×10¹³ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 7×10¹³ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 8×10¹³ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 9×10¹³ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.1×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.2×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.3×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.4×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.5×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.6×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.7×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.8×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.9×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 2×10¹⁴ vg/kg.

In some embodiments, the agent is administered to the patient in a single dose. In some embodiments, the agent is administered to the patient in two or more doses.

### Methods for the Delivery of Exogenous Nucleic Acids to Target Cells

### Transfection Techniques

Techniques that can be used to introduce a transgene, such as a GAA transgene described herein, into a target cell are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Target Genes by Gene Editing Techniques

In addition to the above, a variety of tools have been developed that can be used for the incorporation of a gene of interest into a target cell, such as a human cell. One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Vectors for Delivery of Exogenous Nucleic Acids to Target Cells

### Viral Vectors for Nucleic Acid Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the invention include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Nucleic Acid Delivery

In some embodiments, nucleic acids of the compositions and methods described herein are incorporated into rAAV vectors and/or virions in order to facilitate their introduction into a cell. rAAV vectors useful in the invention are recombinant nucleic acid constructs that include (1) a transgene to be expressed (e.g., a polynucleotide encoding a GAA protein) and (2) viral nucleic acids that facilitate integration and expression of the heterologous genes. The viral nucleic acids may include those sequences of AAV that are required in cis for replication and packaging (e.g., functional ITRs) of the DNA into a virion. In typical applications, the transgene encodes GAA, which is useful for correcting a GAA-deficiency in patients suffering from lysosomal storage disorders, such as Pompe disease. Such rAAV vectors may also contain marker or reporter genes. Useful rAAV vectors have one or more of the AAV WT genes deleted in whole or in part, but retain functional flanking ITR sequences. The AAV ITRs may be of any serotype (e.g., derived from serotype 2) suitable for a particular application. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8 and 9. For targeting muscle cells, rAAV virions that include at least one serotype 1 capsid protein may be particularly useful. rAAV virions that include at least one serotype 6 capsid protein may also be particularly useful, as serotype 6 capsid proteins are structurally similar to serotype 1 capsid proteins, and thus are expected to also result in high expression of GAA in muscle cells. rAAV serotype 9 has also been found to be an efficient transducer of muscle cells. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8 or AAV9 vector encoding a therapeutic protein pseudotyped with a capsid gene derived from AAV serotype 2. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Pharmaceutical Compositions and Routes of Administration

The therapeutic agents described herein may contain a transgene, such as a transgene encoding a lysosomal enzyme (e.g., GAA), and may be incorporated into a vehicle for administration into a patient, such as a human patient suffering from a lysosomal storage disorder (for example, Pompe disease). Pharmaceutical compositions containing vectors, such as viral vectors, that contain the transcription regulatory elements described herein operably linked to a therapeutic transgene can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Viral vectors, such as AAV vectors and others described herein, containing the transcription regulatory element operably linked to a therapeutic transgene may be administered to a patient (e.g., a human patient) by a variety of routes of administration. The route of administration may vary, for example, with the onset and severity of disease, and may include, e.g., intradermal, transdermal, parenteral, intravenous, intramuscular, intranasal, subcutaneous, percutaneous, intratracheal, intraperitoneal, intraarterial, intravascular, inhalation, perfusion, lavage, and oral administration. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Mixtures of the nucleic acids and viral vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Establishing Therapeutic Expression of Acid Alpha-Glucosidase in Mouse Models of Pompe Disease while Avoiding Toxic Side Effects

### Objective

Pompe disease (glycogen storage disease type II, acid maltase deficiency) is an autosomal recessive disorder caused by a deficiency of the lysosomal enzyme acid alpha-glucosidase (GAA). GAA degrades glycogen to glucose within lysosomes. Severely reduced or absent GAA activity results in lysosomal and cytoplasmic glycogen accumulation. This ultimately may lead to death in the more severely affected individuals from impaired cardiac and respiratory function.

The objective of this study was to evaluate the pharmacodynamic response together with potential toxicity of an AAV2/8 vector containing a GAA transgene operably linked to a muscle creatine kinase (MCK) promoter (referred to herein as “AAV2/8-MCK-GAA”) in adult Gaa^{-/-} mice for a period of 12 weeks post dosing.

### Materials and Methods

Seventy-two Gaa−/− mice and 18 wild type littermates were enrolled on study. Eighteen mice (9 each male and female; 10-12 weeks old) per group were administered either a single IV injection (via tail vein) of vehicle or AAV2/8-MCK-GAA (rAAV8-eMCK-hGAA) at doses of 0.3×10¹⁴, 1.0×10¹⁴, or 3.0×10¹⁴ vg/kg. Of these, Cohort-1 animals (5 males and 5 females per dose group plus vehicle controls) were designated for safety evaluation.

Animals were monitored daily for general health status; detailed clinical observations, body weight, and functional assessments occurred weekly. Animals were euthanized at Week 12 or 13 postdosing for clinical pathology and histopathology evaluations. Clinical chemistry, hematology, and anti-GAA antibodies were determined, as well as a comprehensive set of tissues were collected for histopathologic evaluation

### Results

### Mortality

All wild type and AAV2/8-MCK-GAA-treated animals survived until study termination. Two vehicle-treated Gaa−/− mice in Group 2 (1 female, 2506 and 1 male, 2005), were found dead on days 35 and 66 respectively and were sent for full histopathology.

All animals showed a steady gain in body weight during the 12-week course of the study.

### Dose Assurance and Biocompatibility

A dose-assurance analysis was performed to ascertain if the materials used to infuse AAV2/8-MCK-GAA in anyway compromised drug delivery and exposure in this study by evaluating the vector genome titer and the capsid titer of the test article from the flow through.

Dose assurance was performed by holding the drug product, AAV2/8-MCK-GAA within the infusion apparatus for either 0, 3, or 6 hours to mimic the maximal time for potential exposure. Results from the study showed that exposure of the drug product to the administration devices over time had no significant impact on the concentration of the product. These data demonstrated that AAV2/8-MCK-GAA drug product had minimal binding affinity to the exposed device surfaces.

### Clinical Pathology

Vehicle-treated Pompe (Gaa−/−) mice had minimally increased aspartate aminotransferase (AST) relative to wildtype mice. Elevations in AST can be an indication of muscle damage, a phenotypic characteristic of Gaa−/− mice. Following a single IV injection of AAV2/8-MCK-GAA in Gaa−/− mice at doses of 0.3×10¹⁴, 1.0×10¹⁴, or 3.0×10¹⁴ vg/kg with a 3-month observation period, the increases in AST were less pronounced most notably in the 0.3×10¹⁴ vg/kg dose group. This decrease or normalization of AST in treated Gaa−/− mice relative to WT control mice at all AAV2/8-MCK-GAA dose levels was considered a positive effect of the test article.

No clear interpretations could be made from the hematology data due to the limited number of samples that were obtained.

### Immunology

Humoral immunity to human GAA protein was evaluated from serum samples taken at study termination by a qualified ELISA based assay. A summary of the data is presented in Table 2.

Vehicle treated Gaa−/− mice were negative for anti-GAA antibodies at study termination, whereas AAV2/8-MCK-GAA treated mice showed a strong but highly variable anti-GAA response. Low dose (0.3×10¹⁴ vg/kg) AAV2/8-MCK-GAA treated mice had higher overall anti-GAA titers compared to mid and high dose mice. These data demonstrate that administration of AAV2/8-MCK-GAA drives a robust immunological response against the human GAA protein in mice that is sustained out to 12-weeks.

TABLE 2
AAV2/8-MCK-GAA Total anti-GAA Antibodies in Mouse Serum at 12-weeks

| | | | | Screening | | Anti-GAA Titer | |
|---|---|---|---|---|---|---|---|
| Group | Treatment | Dose (vg/kg) | Gender | Interpretation | N | Mean | SD |
| 2 | Vehicle | 0 | Male | Negative | 8 | BLQ | — |
| | | | Female | Negative | 8 | BLQ | — |
| 3 | AAV2/8- | 0.3 × 10¹⁴ | Male | Positive | 4 | 3,840,000 | 3,169,993 |
| | MCK-GAA | | Female | Positive | 4 | 1,792,000 | 512,000 |
| 4 | AAV2/8- | 1.0 × 10¹⁴ | Male | Positive | 4 | 220,000 | 120,000 |
| | MCK-GAA | | Female | Positive | 4 | 440,000 | 240,000 |
| 5 | AAV2/8- | 3.0 × 10¹⁴ | Male | Positive | 4 | 60,800 | 38,400 |
| | MCK-GAA | | Female | Positive | 4 | 280,000 | 80,000 |

### Histopathology

There was no AAV2/8-MCK-GAA-related mortality and there were no AAV2/8-MCK-GAA-related macroscopic findings in males or females. There was an adverse AAV2/8-MCK-GAA test-article-related finding in Pompe (Gaa−/−) mice in the heart which consisted of minimal to mild fibrosis with associated minimal myofiber degeneration/necrosis in the heart of males at ≥3×10¹³ vg/kg (1/5 males at 0.3×10¹⁴ vg/kg, 3/5 males at 1×10¹⁴ vg/kg, and 1/5 males at 3×10¹⁴ vg/kg). Other test article-related microscopic findings included: minimal mononuclear cell infiltrates in the heart of females at ≥1×10¹³ vg/kg and a male at 0.3×10¹⁴ vg/kg; minimal mixed leukocyte infiltration in the heart of one male and one female at 1×10¹⁴ vg/kg; minimal to mild mononuclear cell infiltrates in one or more skeletal muscles of males and females at ≥0.3×10¹⁴ vg/kg. Test article-related microscopic findings in the liver of males at ≥0.3×10¹⁴ vg/kg included decreased vacuolation relative to Pompe vehicle controls, whereas females at ≥0.3×10¹⁴ vg/kg had increased vacuolation relative to Pompe vehicle controls. Positive effects of AAV2/8-MCK-GAA included absence of vacuolation a characteristic of Pompe disease in multiple tissues including: absence of myofiber vacuolation in the quadriceps, triceps, and diaphragm in all animals at 3×10¹⁴ vg/kg, slight decrease in incidence and/or severity of vacuolation of the choroid plexus in the brain at ≥0.3×10¹⁴ vg/kg, slight decrease in severity of neuronal vacuolation in the spinal cord in males at 3×10¹⁴ vg/kg and females at ≥1×10¹⁴ vg/kg, reduction in both severity and incidence of neuronal vacuolation in dorsal root ganglia, a decreased incidence or absence of axonal/myelin degeneration in AAV2/8-MCK-GAA treated animals, a decreased incidence of interstitial cell vacuolation of the prostate and seminal vesicle, and a slight AAV2/8-MCK-GAA treatment-related trend towards decreased incidence and/or severity of vacuolation in the parathyroid gland chief cells.

### GAA Protein and GAA Activity

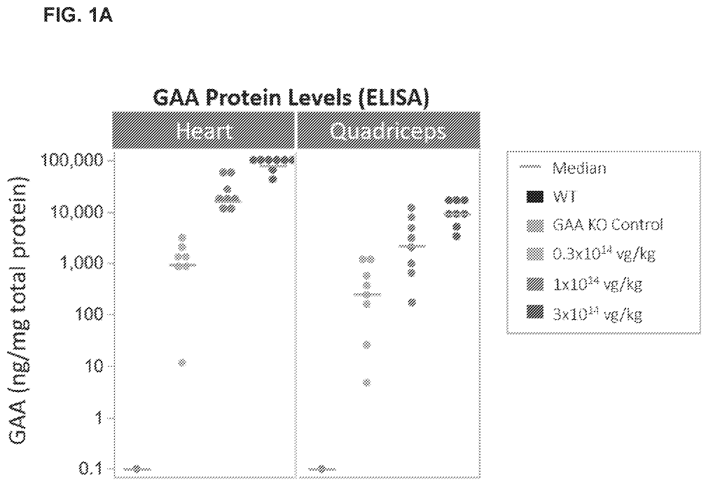

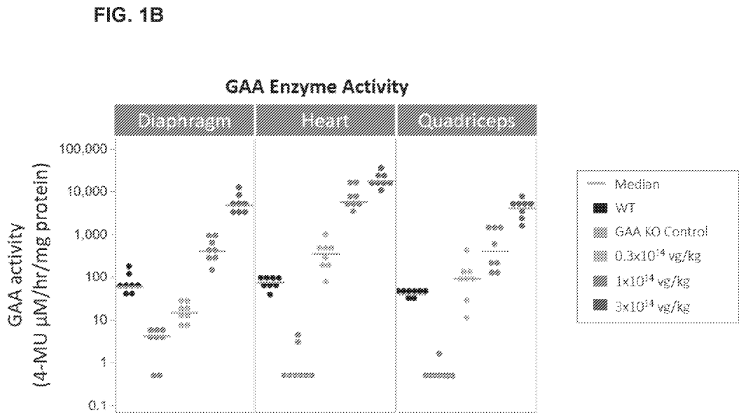

On average, low dose (0.3×10¹⁴ vg/kg) AAV2/8-MCK-GAA-treated mice had higher GAA protein levels in the heart and quadriceps compared to controls. In comparison both to controls and low dose-treated animals, mid (1×10¹⁴ vg/kg) and high dosage (3×10¹⁴ vg/kg) groups had an even greater level of GAA protein in both examined muscles (FIG. 1A). These data showed a dose-dependent increase in GAA expression in muscles following administration of AAV2/8-MCK-GAA.

The same dose-dependent pattern of results was observed in the levels of GAA-enzymatic activity. On average, low dose (0.3×10¹⁴ vg/kg) AAV2/8-MCK-GAA-treated mice had higher GAA-enzyme activity in the diaphragm, heart, and quadriceps compared to controls. Again, in comparison to these groups, mid (1×10¹⁴ vg/kg) and high dose (3×10¹⁴ vg/kg) AAV2/8-MCK-GAA-treated mice had even higher GAA-enzyme activity in all tissues examined (FIG. 1B). These data showed a dose-dependent increase in GAA-enzyme activity in muscles following administration of AAV2/8-MCK-GAA.

### Tissue Glycogen Accumulation

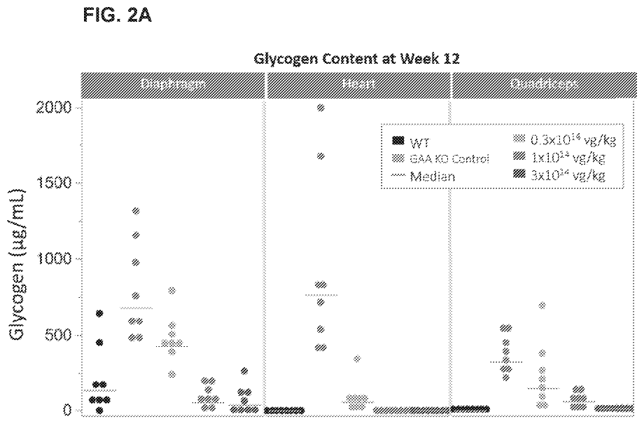

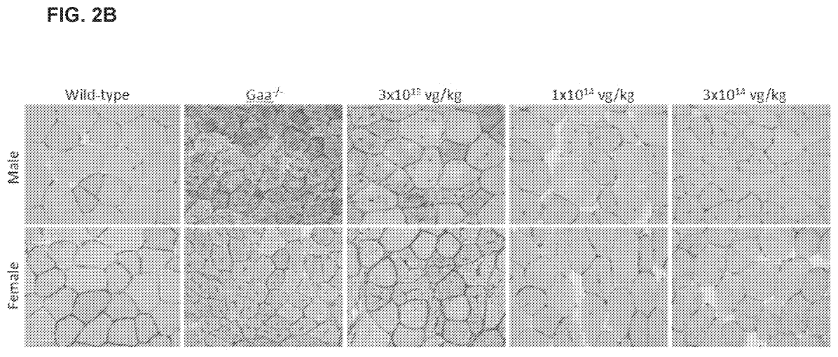

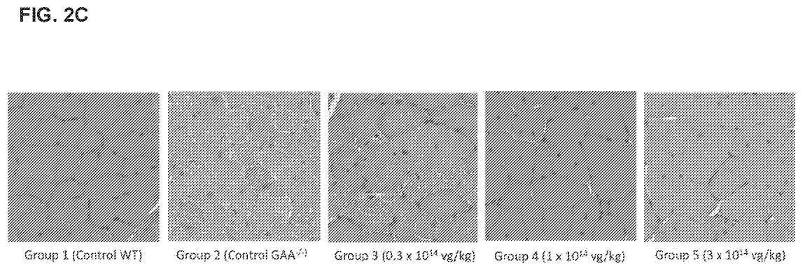

Analysis of diaphragm, heart, and quadricep biopsies revealed a dose-dependent decrease in glycogen concentration in AAV2/8-MCK-GAA-treated mice. Independently of which tissue was tested, the overall pattern of results across muscles was the same. As compared to Vehicle-treated Gaa−/− controls, all three doses of AAV2/8-MCK-GAA elicited a decrease in glycogen, with the highest dose eliciting the greatest reduction. In the heart and quadriceps, specifically, all dosages restored glycogen to levels comparable to Vehicle-treated WT controls (FIG. 2A). This pattern of results was additionally supported by visualization with Periodic Acid Schiff and hematoxylin and eosin (H&E) stainings in the quadriceps (FIGS. 2B and 2C). In the diaphragm, mid and high doses elicited a restoration of glycogen levels comparable to controls. These results revealed robust dose-dependent glycogen clearance in muscles.

### Grip-Response Test:

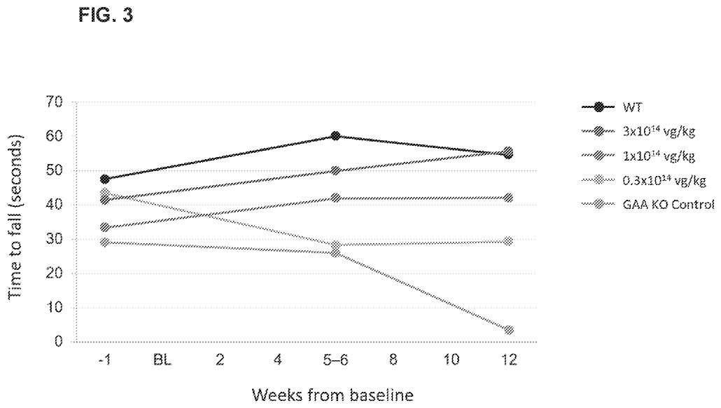

To evaluate the effects of AAV2/8-MCK-GAA treatment on the function of isotonic/isometric muscle strength and sensory motor coordination, a grip-response test was performed. In this assay, the ability of mice to grip onto an inverted wire screen for a 60-second period is evaluated. Using this test, a time-dependent functional correction was observed. Specifically, at 5-6 weeks after Vehicle or AAV2/8-MCK-GAA administration, respectively, the four knockout groups (Vehicle-treated; low, mid, and high AAV2/8-MCK-GAA-treated dosage groups) performed below the level of WT controls (seconds to fall)(FIG. 3). While Gaa−/− Vehicle-treated controls had the worst functional performance, that which was observed in AAV2/8-MCK-GAA-treated mice was superior in a dose-dependent manner. The AV2/8-MCK-GAA high dose-treated group performed closest to WT controls. At 12 weeks from viral vector administration, the performance of the high dosage group was equivalent to WT controls. These results indicate that AAV2/8-MCK-GAA can ameliorate functional motor deficits.

### Conclusions

Early death in vehicle treated Gaa−/− animals may be related to disease progression in this model, likely due to the progressive neurodegeneration and loss of muscle function. A recent study by Keeler, 2019, reported mortality in Gaa−/− mice of the same strain used in this study starting at around 22-weeks of age with ˜50% of Gaa−/− mice dying prematurely by ˜36-weeks of age.

The dose assurance study confirmed compatibility of the dosing devices with the drug product AAV2/8-MCK-GAA without loss due to non-specific binding to the devices. These data confirmed that all doses of AAV2/8-MCK-GAA were successfully administered to Gaa−/− without loss. GAA-protein and enzyme activity levels increased in a dose-dependent manner in all tissues examined.

Clinical pathology showed a positive effect of AAV2/8-MCK-GAA on AST in Gaa−/− mice at all dose levels, but most notably at the lowest dose of 0.3×10¹⁴ vg/kg, thereby normalizing AST in these mice.

Histopathology showed minimal to mild fibrosis in the heart of male Gaa−/− mice in all AAV2/8-MCK-GAA dose groups. Minimal to mild mononuclear cell infiltrates were observed in the heart and skeletal muscles of some AAV2/8-MCK-GAA treated animals. Positive test article effects in reducing intracellular vacuolation were observed in; skeletal muscles, brain, spinal cord, dorsal root ganglia, peripheral axons, male gonads, and parathyroid.

The gender bias observed in the pathology findings of males treated with AAV2/8-MCK-GAA may in part be attributed to the observed higher transduction of AAV that has been reported to occur in male mice compared to females (Davidoff, 2003). Davidoff and colleagues previously showed that AAV particles were 5- to 13-fold higher in male mice than females following systemic delivery of AAV2 or AAV5 vectors.

A positive effect of AAV2/8-MCK-GAA treatment was observed on glycogen clearance across all tested dosage groups and in all examined tissues. The greatest improvement was observed in the mid and high dose treatment groups, with glycogen normalized to the level observed in WT controls.

AAV2/8-MCK-GAA treatment also mediated a functional correction of grip. In a time- and dose-dependent manner, at 12 weeks after AAV2/8-MCK-GAA administration a complete functional restoration was observed in the high dose group.

### Example 2. Establishing Therapeutic Expression of Acid Alpha-Glucosidase in Mouse Models of Pompe Disease while Avoiding Toxic Side Effects

### Objective

The objective of this GLP study was to examine the potential toxicity and safety pharmacology of AAV2/8-MCK-GAA in juvenile cynomolgus monkeys for a period of 12 weeks post-dosing.

### Materials and Methods

Twenty-five juvenile monkeys 2-4 years old (14 males, 11 females) with low serum anti-AAV8 neutralizing antibody levels (titer of 5 or less for AAV2/8-MCK-GAA treated) were enrolled on study. Table 3 outlines the study design. Animals were administered a single IV infusion of vehicle, one of three doses (0.6×10¹⁴, 2×10¹⁴, or 5×10¹⁴ vg/kg) of AAV2/8-MCK-humanGAA (AAV2/8-MCK-GAA), or one dose (2×10¹⁴ vg/kg) of AAV2/8-MCK-cynomolgus GAA on study Day 1.

TABLE 3
Study Design

| | | | Dose | Dose | Dose | Number of | |
|---|---|---|---|---|---|---|---|
| | Animal | | Level | Volume | Concentration | Animals | |
| Group | Nos. | Test Material | (vg/kg/day) | (ml/kg) | (vg/mL) | Males | Females |
| 1 | 1001, | Vehicle | 0 | 23 | 0 | 2 | 2 |
| | 1002; | | | | | | |
| | 1501, | | | | | | |
| | 1502 | | | | | | |
| 2 | 3001, | AAV2/8-MCK- | 0.6 ×× 10¹⁴ | 23 | 2.6 × 10¹² | 3 | 3 |
| | 3002, | GAA | | | | | |
| | 3003; | | | | | | |
| | 3501, | | | | | | |
| | 3502, | | | | | | |
| | 3503 | | | | | | |
| 3 | 3001, | | 2.0 ×× 10¹⁴ | 23 | 0.86 × 10¹³ | 3 | 3 |
| | 3002, | | | | | | |
| | 3003; | | | | | | |
| | 3501, | | | | | | |
| | 3502, | | | | | | |
| | 3503 | | | | | | |
| 4 | 4001, | | 5.0 ×× 10¹⁴ | 23 | 2.16 × 10¹³ | 3 | 3 |
| | 4002, | | | | | | |
| | 4003; | | | | | | |
| | 4501, | | | | | | |
| | 4502, | | | | | | |
| | 4503 | | | | | | |
| 5 | | AAV2/8-MCK- | 2.0 ×× 10¹⁴ | | | 3 | 0 |
| | | cynomolgusGAA | | | | | |

Animals were monitored daily for general health status. Detailed clinical observations and body weight assessments were conducted at least weekly. Ophthalmic examinations were conducted predose and during Weeks 4 and 12. ECG waveforms were collected using jacketed external telemetry predose and during Weeks 4, 8, and 12; data were acquired using Ponemah software (Version 5.0). Parameters derived from the ECG waveforms included heart rate and the RR, PR, QRS and QT intervals. QT was corrected for each animal based on individual correction factors determined from the predose data (QT_{ca}). Qualitative evaluation of ECG tracings was conducted by a board-certified veterinary cardiologist. Blood pressure was assessed predose and during Weeks 3, 4, 8, and 12. Echocardiograms were conducted predose and during Weeks 4, 8, and 12 and assessed by a board-certified veterinary cardiologist. A neurologic battery (general attitude, behavior, motor function, cranial nerves, proprioception and postural reactions, and spinal nerves) was conducted predose and during Weeks 4, 8, and 12.

Clinical pathology samples were collected for assessment of hematology, coagulation, clinical chemistry, urinalysis, and cardiac biomarkers (Troponin-I, BNP, as well as CK-MB, CK-MM, CK-BB) at predose and Days 3 (hematology only), 7, 14, 21, 28, 56, 73, and 84. Bioanalytical samples were collected for assessment of anti-AAV8 neutralizing antibodies (NAbs) at baseline and anti-GAA total IgG antibodies (TAbs) at baseline and Days 14, 35, and 84. Whole blood was also collected for peripheral blood mononuclear cell (PBMC) isolation and evaluation of T-cell response to human GAA and AAV8 capsid at baseline, Days 28 and 84.

Animals were euthanized on Day 85. All animals had a complete necropsy examination, and weights of selected organs were recorded. A comprehensive panel of tissues were collected for histopathologic evaluation.

### Results

### Mortality

Two animals from the high dose group (5×10¹⁴ vg/kg), a male (animal 4003) and a high dose female (animal 4501) underwent unscheduled necropsy on Day 82 and 79 respectively. Twenty remaining animals, 10 males and 10 females survived until scheduled terminal euthanasia on Day 85.

### Clinical Observations

There were no AAV2/8-MCK-GAA-related clinical signs in 20 of 22 animals that survived until study termination. Clinical signs were apparent in the two animals (male 4003 and female 4501) in the high dose (5.0×10¹⁴ vg/kg) group during the week prior to study termination.

For male 4003, there were no concerning clinical signs observed but due to echocardiograph finding concerns this animal was sent to early euthanasia, 3 days ahead of schedule.
For female 4501, there were no clinical signs observed until Day 77. On day 78 the animal was hunched and weak. On Day 79 after echocardiograph recording the animal was cold to touch, lying on its side, and unable to recover from anesthesia. Due to rapidly declining condition the animal was euthanized on Day 79, 6 days ahead of schedule.

All clinical observations that occurred in the remaining animals on study were considered unrelated to test articles because they occurred across all dose groups including the controls, were isolated occurrences, were considered procedurally related, and/or were common incidental findings in laboratory housed cynomolgus monkeys undergoing similar study procedures. These clinical signs included skin discoloration, abrasions, and bruising.

### Body Weights

There were no AAV2/8-MCK-GAA-related effects on body weight except the week prior to necropsy. However, these changes occurred across all dose groups and were not considered to be test article related.

All animals either gained or maintained weight for the duration of the study. Fluctuations in body weights were considered incidental and/or were of a magnitude of change commonly observed in cynomolgus monkeys.

### Safety Pharmacology Assessments

Neurological Assessments

There were no AAV2/8-MCK-GAA-related changes in the neurological assessments for the duration of the study.

ECG and Heart Rate by Jacketed External Telemetry

Qualitatively, no AAV2/8-MCK-GAA-related abnormalities in rhythm or waveform morphology were found at any dose level based on comparison of predose and post dose electrocardiographic recordings. There was a generalized non-dose responsive elevation in heart rate in all AAV2/8-MCK-GAA treated groups that was not considered adverse.

Echocardiograms

Doses of ≥2.0×10¹⁴ vg/kg of AAV2/8-MCK-GAA showed adverse changes in cardiac function by echocardiography that led to the early termination of two animals, a female at Day 79 and male at Day 82. Some animals in the low dose of 0.6×10¹⁴ vg/kg of AAV2/8-MCK-GAA showed slight but meaningful differences in echocardiographic parameters at Day 79 compared to pre-dose. One animal (2003) in the low dose (0.6×10¹⁴ vg/kg) group had a pre-existing pulmonic regurgitation that was not adversely affected by AAV2/8-MCK-GAA. It is uncertain if these represent sporadic biologic variations within cynomolgus or fluctuations associated with individual variations in the depth of anesthesia (Sleeper, 2008).

### Hematology

In hematology parameters, no notable changes were observed. Total white blood cell counts were highly variable, but no specific pattern was noted to indicate AAV2/8-MCK-GAA-related changes.

Differences noted in hematology parameters were not considered AAV2/8-MCK-GAA related and were attributed to biologic variation because they were sporadic, similar to fluctuations in control and/or pre-study values, and/or were of a magnitude of change commonly observed in cynomolgus monkeys under similar study conditions.

### Coagulation

In coagulation, no changes were noted with administration of AAV2/8-MCK-GAA at any dose.

Differences noted in coagulation parameters were not considered AAV2/8-MCK-GAA related and were attributed to biologic variation because they were sporadic, similar to fluctuations in control and/or pre-study values, and/or were of a magnitude of change commonly observed in cynomolgus monkeys under similar study conditions.

### Clinical Chemistry

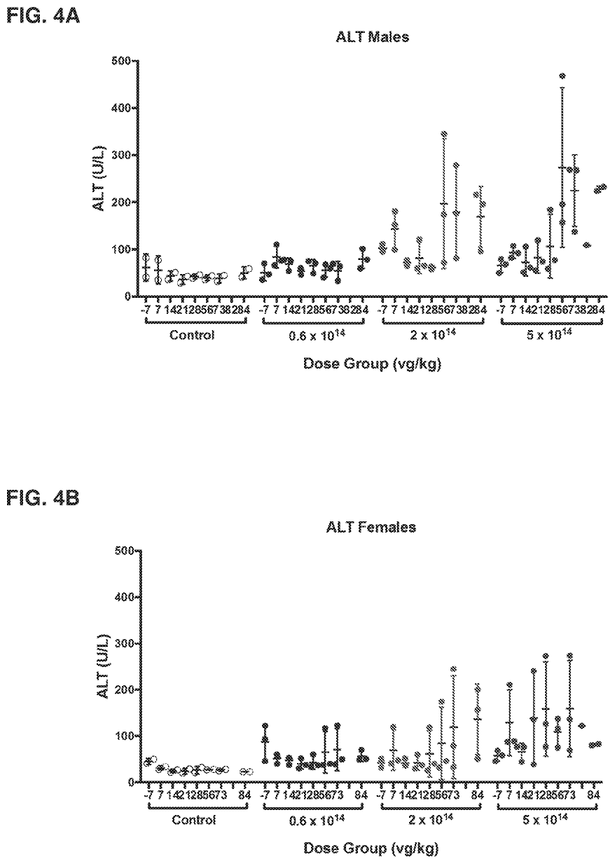

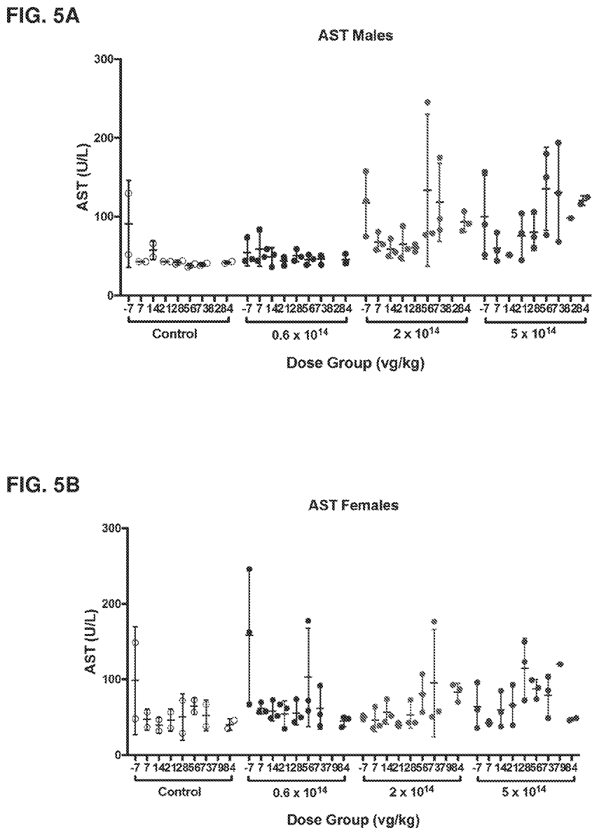

In clinical chemistry, temporal-dependent minimal to mild increases in alanine aminotransferase (ALT) activity was noted. ALT was transiently increased in all AAV2/8-MCK-GAA dose groups males and mid (1×10¹⁴ vg/kg) and high dose (5×10¹⁴ vg/kg) groups females on Day 7, decreased on Day 14, and then variably increased to Day 84. Aspartate aminotransferase (AST) activity was minimally increased but with lesser magnitude and fewer timepoints than ALT. Increases in ALT and AST were likely related to inflammation reported on histopathology in skeletal and cardiac muscle with minimal, if any, contribution from hepatocellular effects. No other indicators of hepatocellular effects were noted. ALT and AST values were variable and dose-dependent in males but not females (FIGS. 4A, 4B, 5A, and 5B).

Also noted were minimal to mild decreases in albumin, minimal to mild increases in globulin, and minimal decreases in AGR on Days 56 to 84 primarily in males but also noted in AAV2/8-MCK-GAA high dose (5×10¹⁴ vg/kg) females, suggestive of a minimal acute phase response with no other obvious indicators noted. Triglyceride values were mildly to moderately increased at most timepoints in males and occasional timepoints in females. This could be associated with an acute phase response but also other possibilities since there were potential hepatocellular effects during the study. Urea nitrogen was minimally to mildly increased at numerous timepoints but without concomitant increases in creatinine; therefore, this was considered prerenal. Calcium concentrations were minimally to mildly decreased during the study and likely a result of decreased albumin concentrations since albumin is a systemic carrier of calcium. Phosphorus was mildly increased (+48%) in high dose (5×10¹⁴ vg/kg females) on Day 84 but the cause of this is uncertain and the values at this timepoint were lower than highest pre-study value.

Remaining differences in clinical chemistry parameters were not considered AAV2/8-MCK-GAA related and were attributed to biologic variation because they were sporadic, similar to fluctuations in control and/or pre-study values, and/or were of a magnitude of change commonly observed in cynomolgus monkeys under similar study conditions.

Cardiac Biomarkers

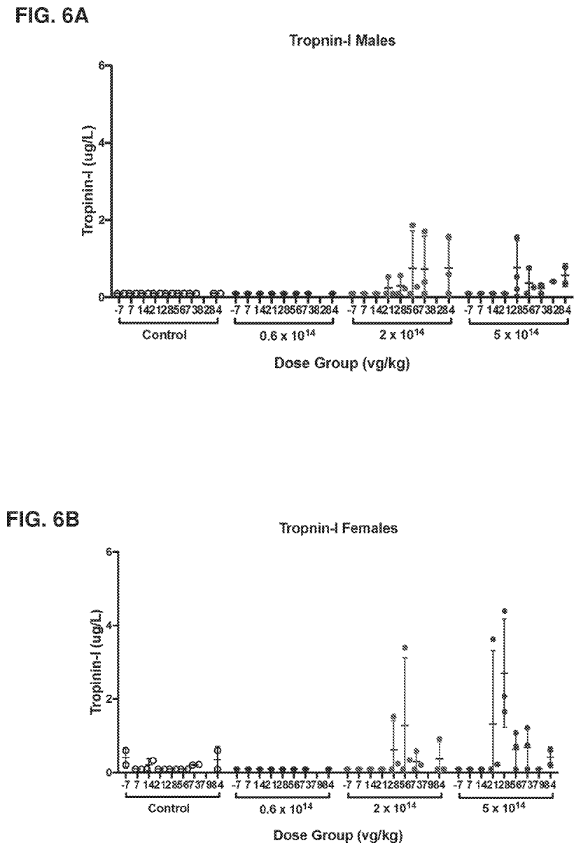

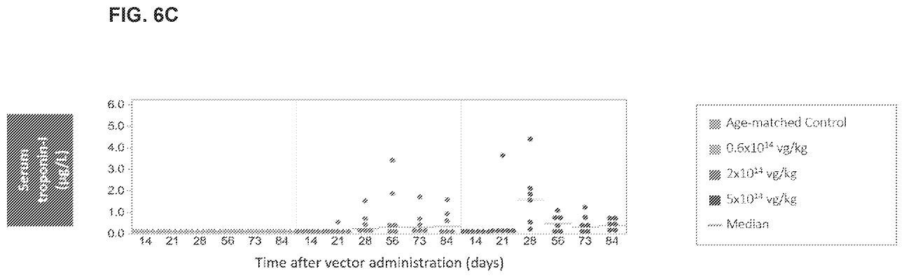

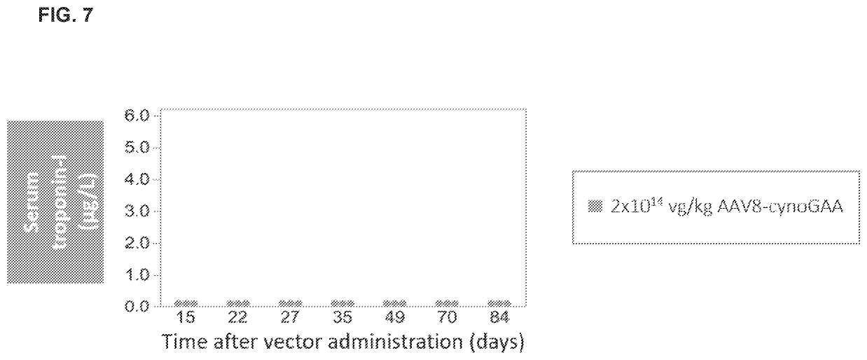

No changes in Troponin-I were noted in vehicle-treated (Group 1), low dose (0.6×10¹⁴ vg/kg) AAV2/8-MCK-GAA-treated (Group 2), or AAV2/8-MCK-cynomolgusGAA-treated (Group 5) animals (FIGS. 6A, 6B, 6C and 7). Troponin-I was minimally to markedly increased in mid (2.0×10¹⁴ vg/kg) and high dose (5.0×10¹⁴ vg/kg) in both sexes, with notable increases beginning on Day 21 in some animals and all animals by Day 56. Peak values were generally noted on Days 21 to 56 with high dose male 4001 peaking on Day 84. After peak values, most animals incrementally decreased on Day 73 but some remained similar until Day 84. Increases in Troponin-I indicated myocardial injury and corresponded with inflammation noted in cardiac muscle histopathology. Generally, increased Troponin-I corresponded with notable increases in BNP but did not correspond with any notable changes in CK-MB.

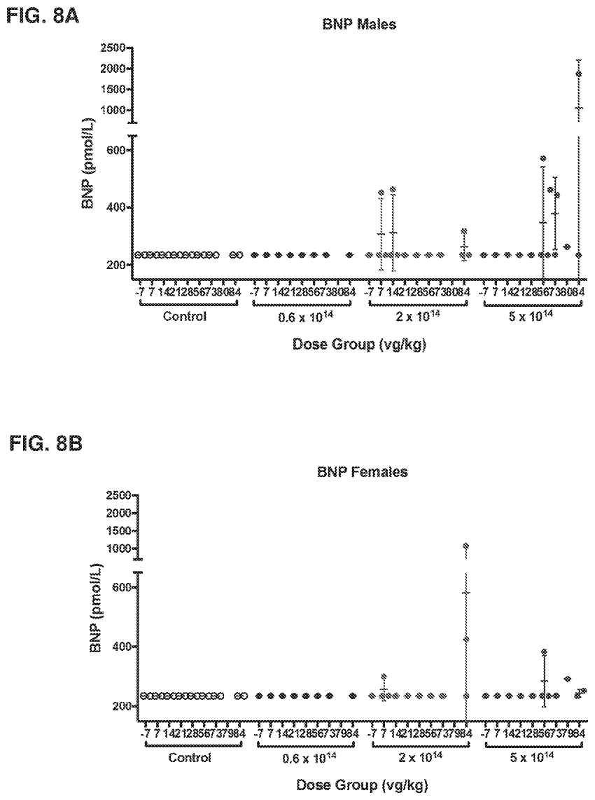

Brain natriuretic peptide (BNP) values were markedly increased (FIGS. 8A and 8B) in mid dose female 3501 (+363%) and a high dose male 4001 (+697%) on Day 84. While creatine kinase (CK) was minimally higher in the high dose male 4001, creatine kinase-MB (CK-MB) was not increased in either animal to indicate any cardiac muscle effects at this timepoint when compared with pre-study values. In addition, high dose male 4003 had a minimal increase in BNP on Day 56 but by unscheduled Day 82 the BNP value had lessened and was only minimally higher than pre-study low measurement limit. A mid dose male 3003 had a minimal increase on Days 7 and 14 lessening to pre-study by Day 21 and then minimally increased on Day 84 (+92%, +97%, and +35%, respectively). A mid dose female 3503 had a minimal increase in BNP on Day 84 (+81%). Female 3501 had a minimal increase in urea nitrogen (+230%) without a concomitant increase in creatinine on Day 84. Increases in BNP in these animals, generally corresponded with increases in Troponin-I and was indicative of myocardial injury and corresponded with myocardial inflammation noted on histopathology. No obvious increases in CK-MM, CK-MB, or CK-BB were noted. Variability did occur but this variability also was noted within the control group.

### Urinalysis

In urinalysis parameters, no changes were noted after administration of AAV2/8-MCK-GAA at any dose.

Differences in urinalysis parameters were not considered AAV2/8-MCK-GAA related and were attributed to biologic variation or contaminant interference because they were sporadic, similar to fluctuations in control and/or pre-study values, lacked correlative changes in other clinical pathology parameters, and/or were of a magnitude of change commonly observed in cynomolgus monkeys under similar study conditions.

### Dose Assurance and Biocompatibility

A dose-assurance analysis was performed to ascertain if the materials used to infuse AAV2/8-MCK-GAA in anyway compromised drug delivery and exposure in this GLP study by evaluating the vector genome titer and the capsid titer of the test article from the flow through.

Dose assurance was performed by holding the drug product AAV2/8-MCK-GAA within the infusion apparatus for either 0, 3, or 6 hours, to mimic the maximal time for potential exposure. Results from the study show that exposure of the drug product to the administration devices over time had no significant impact on the concentration of the product. These data demonstrated that AAV2/8-MCK-GAA drug product had minimal binding affinity to the exposed device surfaces and accurate dosing was assured.

### GAA Protein and GAA Activity

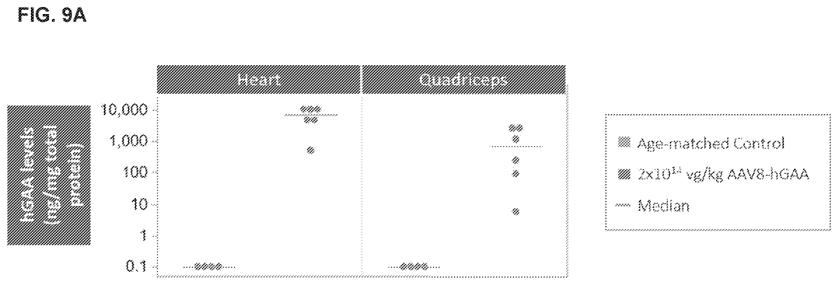

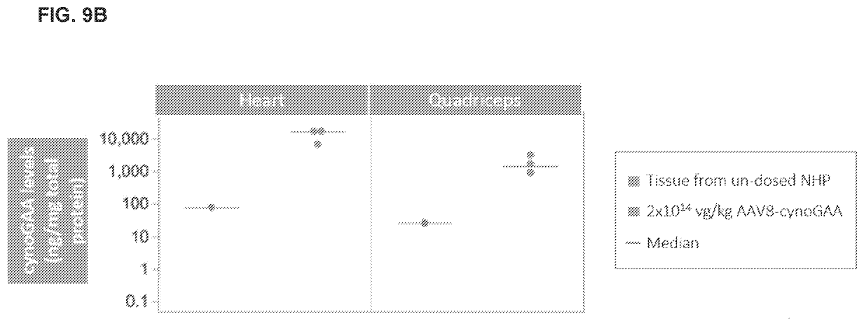

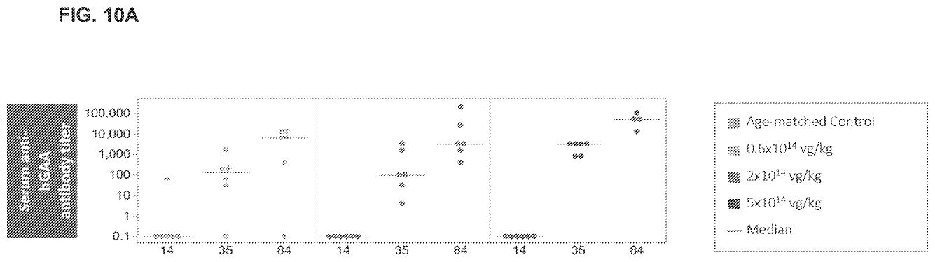

On average, low dose (0.6×10¹⁴ vg/kg) AAV2/8-MCK-GAA-treated animals had ˜1-2-fold higher human GAA-enzyme activity in the quadriceps compared to controls, and ˜2-5-fold higher in the heart. Mid-dose (2.0×10¹⁴ vg/kg) had ˜4 2-52-fold higher human GAA in quadriceps and ˜84-89-fold higher human GAA-activity in the heart. In contrast high dose (5.0×10¹⁴ vg/kg) animals on average had ˜53-69 higher human GAA-activity in quadriceps and ˜47-88-fold higher human GAA-activity in the heart (FIG. 9A). These data showed a dose-dependent increase in human GAA-enzyme protein expression and activity following administration of AAV2/8-MCK-GAA.

On average, AAV2/8-MCK-cynomolgusGAA-treated animals had higher cynomolgus GAA in the quadriceps and heart compared to controls (FIG. 9B).

### Immunology

Anti-AA V8 Antibodies

At baseline, pre-study, animals were assessed for anti-AAV8 neutralizing antibodies (NAb) to guide randomization into treatment groups so that AAV2/8-MCK-GAA-treated animals had titers of 5 or less and vehicle treated animals had titers of 10 or >20.

Humoral Response to hGAA

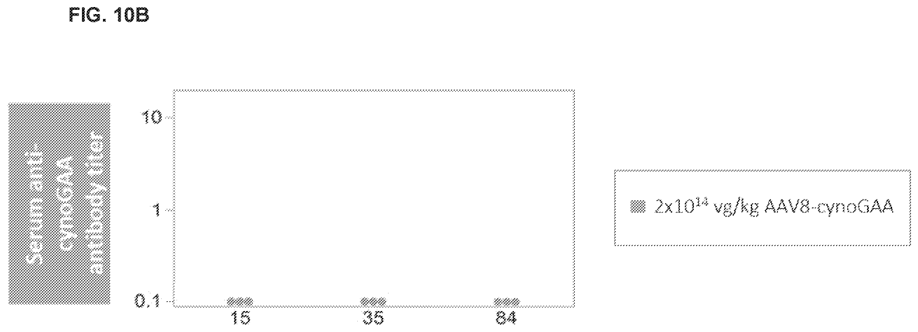

Humoral immunity to human or cynomolgus GAA protein was evaluated from serum samples taken pre-dose, 14 or 15, 35, and 84-days post-dosing by a qualified ELISA based assay. A summary of the data regarding human GAA protein response to hGAA is presented in FIG. 10A and Table 4.

All predose and Day 14 animals had anti-GAA titers <80. There was a dose-dependent increase in anti-GAA titers in all AAV2/8-MCK-GAA-treated animals at Day 35 (range 349-2,400), and a more robust increase at Day 84 (range 7,760-54,400). There was a significant difference in anti-GAA titers in males and females at Day 35 and Day 84, with males having higher mean titers. These data demonstrate that intravenous delivery of AAV2/8-MCK-GAA targeting muscle cell expression of GAA, results in systemic exposure that triggers a humoral immune response to human GAA in cynomolgus monkeys.

TABLE 4
AAV2/8-MCK-GAA Anti-GAA Antibodies in Cynomolgus Serum

| | Treatment | Anti-GAA Titer | | | | | | |
|---|---|---|---|---|---|---|---|---|
| | Dose | | Male | | | Female | | |
| Group | (vg/kg) | Day | n +ive | Mean | SD | n +ive | Mean | SD |
| 1 | Vehicle | Predose | 0/2 | — | — | 0/2 | — | — |
| | | 14 | 0/2 | — | — | 0/2 | — | — |
| | | 35 | 0/2 | — | — | 0/2 | — | — |
| | | 84 | 0/2 | — | — | 0/2 | — | — |
| 2 | 0.6 x 10¹⁴ | Predose | 1/3 | 64 | — | 1/3 | 80 | — |
| | | 14 | 1/3 | 64 | — | 3/3 | — | — |
| | | 35 | 3/3 | 610.67 | 860.90 | 3/3 | 88 | 102.14 |
| | | 84 | 3/3 | 6,533.33 | 6,201.08 | 2/3 | 9,600 | 4,525.48 |
| 3 | 2.0 x 10¹⁴ | Predose | 3/3 | — | — | 3/3 | — | — |
| | | 14 | 3/3 | — | — | 3/3 | — | — |
| | | 35 | 3/3 | 1,101.33 | 1,818.13 | 3/3 | 577.33 | 886.31 |
| | | 84 | 3/3 | 68,933.33 | 117,665.51 | 3/3 | 10,666.66 | 12,932.64 |
| 4 | 5.0 x 10¹⁴ | Predose | 3/3 | — | — | 3/3 | — | — |
| | | 14 | 3/3 | — | — | 3/3 | — | — |
| | | 35 | 3/3 | 2,400 | 1,385.64 | 3/3 | 2,400 | 1,385.64 |
| | | 84 | 2/3 | 76,800 | 36,203.86 | 2/3 | 32,000 | 27,152.90 |

kg = kilogram;
N = number;
+ive = positive samples;
SD = standard deviation;
vg = vector genomes

Humoral immunity to cynomolgus GAA protein was evaluated from serum samples taken 15, 35, and 84-days post-dosing by a qualified ELISA based assay. All animals had anti-cynomolgus GAA titers <1. Over time, no change in anti-GAA titers were observed (FIG. 10B). These results indicate that the cynomolgus GAA protein did not elicit an immune response.

Cellular Immune Response to AAV8 and hGAA

Cellular immunity to AAV8 capsid peptide pools (A, B, C) and human GAA peptide pools (A, B, C, and D) were evaluated from isolated peripheral blood mononuclear cell (PBMC) samples taken pre-dose, 28, and 84-days post-dosing using a qualified IFN-gamma ELISPOT assay. In isolated PBMCs, peripheral T-cell responses were observed as spot-forming units (SFUs) as early as Day 28 at all dose levels. Summarized data are presented in Table and Table 6.

Some vehicle treated animals showed positive T-cell responses to AAV8 capsid peptide pools consistent with their known prescreening AAV8 NAb titers. None of the animals assigned to the AAV2/8-MCK-GAA treatment groups showed positive responses at predose (Table). All low dose (0.6×10¹⁴ vg/kg) treated animals remained negative for T-cell responses to AAV8 at Days 28 and 84. Mid dose (2.0×10¹⁴ vg/kg) treated animals showed positive ELISpot signals in 1/3 males and 2/3 females at Day 28 but not at Day 84. At the high dose (5.0×10¹⁴ vg/kg) males showed positive T-cell responses at Day 28 but not 84, whereas females showed positive signals at both 28 and 84-days. These data suggest that when T-cell responses to AAV8 are present, they most frequently occur at Day 28, particularly when the vector load is high ≥2.0×10¹⁴ vg/kg.

All vehicle treated animals were negative for hGAA T-cell responses for the duration of the study. At the low dose (0.6×10¹⁴ vg/kg) 1/3 males had positive SFUs to pools A, C, and D at Day 28 increasing to 2/3 males by Day 84. 0/3 low dose females showed a positive ELISpot signal. At the mid dose (2.0×10¹⁴ vg/kg) 1/3 males and 1/3 females had positive SFU's at Day 28, and at Day 84 2/3 males and 1/3 females were positive. At the high dose (5.0×10¹⁴ vg/kg) 1/3 males and 1/3 females had positive ELISpot signals at Day 28, with up to 3/3 males and 2/3 females having positive SFUs by Day 84. The only consistent pattern in the T-cell responses to GAA was reactivity to peptide pools A and D by Day 84 in the mid and high dose group animals. Reactivity to pools B and C varied between males and females.

TABLE 5
AAV2/8-MCK-GAA IFN-gamma AAV8 T-cell Responses from
Cynomolgus PBMCs

| | | T-cell Responses to AAV8 Peptide Pools | | | | | | |
|---|---|---|---|---|---|---|---|---|
| | | | Male | | | Female | | |
| | Treatment | | N Positive/Total | | | N Positive/Total | | |
| Group | Dose (vg/kg) | Day | A | B | C | A | B | C |
| 1 | Vehicle | Predose | 1/2 | 1/2 | 0/2 | 0/2 | 1/2 | 0/2 |
| | | 28 | 1/2 | 0/2 | 0/2 | 2/2 | 1/2 | 0/2 |
| | | 84 | 0/2 | 0/2 | 0/2 | 0/2 | 1/2 | 0/2 |
| 2 | 0.6 × 10¹⁴ | Predose | 0/3 | 0/3 | 0/3 | 0/3 | 0/3 | 0/3 |
| | | 28 | 0/3 | 0/3 | 0/3 | 0/3 | 0/3 | 0/3 |
| | | 84 | 0/3 | 0/3 | 0/3 | 0/3 | 0/3 | 0/3 |
| 3 | 2.0 × 10¹⁴ | Predose | 0/3 | 0/3 | 0/3 | 0/3 | 0/3 | 0/3 |
| | | 28 | 1/3 | 0/3 | 0/3 | 2/3 | 2/3 | 1/3 |
| | | 84 | 0/3 | 0/3 | 0/3 | 0/3 | 0/3 | 0/3 |
| 4 | 5.0 × 10¹⁴ | Predose | 0/3 | 0/3 | 0/3 | 0/3 | 0/3 | 0/3 |
| | | 28 | 1/3 | 3/3 | 1/3 | 1/3 | 1/3 | 0/3 |
| | | 84 | 0/3 | 0/3 | 0/3 | 1/3 | 2/3 | 1/3 |

A, B, C = AAV8 peptide pools;
Kg = kilogram;
N = number;
SD = standard deviation;
vg = vector genomes

TABLE 6
AAV2/8-MCK-GAA IFN-gamma GAA T-cell Responses from
Cynomolgus PBMCs

| | | T-cell Responses to hGAA Peptide Pools | | | | | | | | |
|---|---|---|---|---|---|---|---|---|---|---|
| | Treatment | | Male | | | | Female | | | |
| | Dose | | N Positive/Total | | | | N Positive/Total | | | |
| Group | (vg/kg) | Day | A | B | C | D | A | B | C | D |
| 1 | Vehicle | Predose | 0/2 | 0/2 | 0/2 | 0/2 | 0/2 | 0/2 | 0/2 | 0/2 |
| | | 28 | 0/2 | 0/2 | 0/2 | 0/2 | 0/2 | 0/2 | 0/2 | 0/2 |
| | | 84 | 0/2 | 0/2 | 0/2 | 0/2 | 0/2 | 0/2 | 0/2 | 0/2 |
| 2 | 0.6 × 10¹⁴ | Predose | 0/3 | 0/3 | 0/3 | 0/3 | 0/3 | 0/3 | 0/3 | 0/3 |
| | | 28 | 1/3 | 0/3 | 1/3 | 1/3 | 0/3 | 0/3 | 0/3 | 0/3 |
| | | 84 | 2/3 | 0/3 | 2/3 | 2/3 | 0/3 | 0/3 | 0/3 | 0/3 |
| 3 | 2.0 × 10¹⁴ | Predose | 0/3 | 0/3 | 0/3 | 0/3 | 0/3 | 0/3 | 1/3 | 0/3 |
| | | 28 | 0/3 | 1/3 | 0/3 | 0/3 | 1/3 | 0/3 | 0/3 | 1/3 |
| | | 84 | 1/3 | 0/3 | 2/3 | 2/3 | 1/3 | 0/3 | 1/3 | 1/3 |
| 4 | 5.0 × 10¹⁴ | Predose | 0/3 | 0/3 | 0/3 | 0/3 | 0/3 | 0/3 | 0/3 | 0/3 |
| | | 28 | 0/3 | 1/3 | 1/3 | 1/3 | 0/3 | 0/3 | 0/3 | 2/3 |
| | | 84 | 3/3 | 1/3 | 2/3 | 1/3 | 2/3 | 2/3 | 0/3 | 2/3 |

A, B, C, D = hGAA peptide pools;
kg = kilogram;
N = number;
+ive = positive samples;
SD = standard deviation;
vg = vector genomes

### Gross Pathology

No test article-related gross findings were noted. Multifocal dark red discoloration in the lungs of one male (Animal No. 2002) given 0.6×10¹⁴ vg/kg, with correlative regionally extensive minimal alveolar hemorrhage and mild pigmented alveolar macrophages were considered background change, most likely associated with lung mites (Pneumonyssus sp.), and unrelated to administration of AAV2/8-MCK-GAA.

### Organ Weights

No test article-related organ weight changes were noted. There were isolated organ weight values (absolute and/relative) that were different from their respective controls. However, there were no patterns, trends, or correlating data to suggest these values were toxicologically relevant. Thus, the organ weight differences observed were considered within the normal range of biologic variability, and therefore unrelated to administration of AAV2/8-MCK-GAA.

### Histopathology

Tissues for histopathology were stained with both H&E as well as Masson's Trichrome. The predominant AAV2/8-MCK-GAA-related microscopic finding observed in both the main study and early mortality animals, at generally 2×10¹⁴ vg/kg in males and females, consisted of mixed cell interstitial inflammation with amphophilic interstitial granular material and occasional degeneration of myofibers in heart, skeletal and smooth muscle, mixed cell inflammation in the liver, mixed cell inflammation of adipose tissue with rare degeneration of brown fat cells, and gliosis with rare neuronal degeneration of dorsal root ganglia. Findings are discussed with respect to the major organ systems.

Heart, Skeletal, and Smooth Muscle

Alterations in the heart, skeletal muscle (diaphragm, esophagus, biceps brachii, pectoralis major muscle—attached to sternum, vagina—attached skeletal muscle, levator ani and external anal sphincter-attached to rectum, triceps brachii, quadriceps, retrobular—rectus and palpebra muscles of the eye, brachialis muscle at the intravenous administration site), and rare smooth muscle (tunica muscularis of the esophagus and rectum), consisted of minimal to marked mixed cell interstitial inflammation characterized by variable numbers of lymphocytes, plasma cells, histiocytes with interstitial amphophilic granular material and occasional degeneration of myofibers. These changes were observed generally 2×10¹⁴ vg/kg for males and females, with the exception of the rectum where the changes were mainly limited to males at the same dose range, and the triceps brachii and quadriceps muscles in which changes were similar across all dose groups, but more frequent and severe in males. Changes in the heart were most profound in the ventricles but were also evident in the atria. Changes in the skeletal muscle were more severe in the diaphragm, esophagus, rectum, and sternum (attached muscle), with occasional regeneration of myofibers and wrapping in individual myofibers by inflammatory cells; smooth muscle was equally affected in the esophagus and generally less severe, as compared to skeletal muscle in the rectum (colorectal junction). The Masson's Trichrome Stain did not demonstrate increased staining, as compared to respective controls for submitted sections of heart, indicating an absence of increased collagen associated with the degeneration/necrosis of affected cardiomyocytes discussed above.

Minimal inflammatory infiltrates in the myocardium in one control female (animal 1502) was considered an incidental finding, as focal, low numbers of lymphocytic/histiocytic/plasmacytic infiltrates in heart have been reported as common background findings in cynomolgus monkeys (Chamanza, 2010; Chamanza, 2006; Gaillot-Drevon, 2006). These changes are usually characterized by focal interstitial distribution of idiopathic inflammatory infiltrates with minimal to mild degeneration or necrosis of myocytes; in the heart these infiltrates are generally limited to the sub-endocardium or sub-epicardium, as was also seen in the one control female in this study.

Liver

Changes in the liver were generally confined to males at ≥0.6×10¹⁴ vg/kg and consisted of minimal to moderate mixed cell inflammation characterized by variable numbers of lymphocytes and histiocytes, and lesser plasma cells and neutrophils, all arranged in a general portal (and rare subcapsular) distribution that often disrupted the hepatic plate, with separated individual hepatocytes, and occasional individual cell necrosis and a bridging inflammatory pattern (female, Animal 3503).

Adipose Tissue

Changes in fat tissue, generally at 2×10¹⁴ vg/kg/day for both males and females, consisted of minimal to moderate mixed cell (lymphocytes, histiocytes, and plasma cells) inflammation and occasional degeneration of adipocytes characterized by poorly microvacuolated to irregular shaped adipocytes with disrupted pannicles observed in brown fat adjacent to esophagus, thyroid gland, sternum, thymus, heart, aorta, and kidney; this change was most pronounced in the thyroid gland and thymus. Minimal mixed cell inflammation in pericardial white fat was also noted in one high-dose male (animal 4002).

Dorsal Root Ganglia

Changes in the dorsal root ganglia consisted of dose-independent minimal to mild gliosis (mononuclear cells), with occasional degeneration of individual neurons.

Remaining microscopic findings observed were considered incidental, of the nature commonly observed in cynomolgus monkeys (Sato, 2012; Chamanza, 2010; Chamanza, 2006; Gaillot-Drevon, 2006), and/or were of similar incidence and severity in control and dosed animals and, therefore, were considered unrelated to administration of AAV2/8-MCK-GAA.

### Conclusion

Administration of AAV2/8-MCK-GAA by intravenous infusion was tolerated up to 0.6×10¹⁴ vg/kg, but at the highest dose of 5×10¹⁴ vg/kg resulted in the unscheduled euthanasia of two animals, one female (animal 4501) on Day 79 and one male (animal 4003) on Day 82. Dose assurance confirmed test article dosing was unaffected by binding to the infusion apparatus. Bioanalytical data confirmed expression of GAA mRNA that translated into GAA-protein, and functional GAA enzyme activity was evidenced in all animals at all dose groups. GAA-protein and enzyme activity levels as well as anti-GAA total antibodies, increased in a dose-responsive manner in all tissues examined and appeared to be higher in males than females. Furthermore, in AAV2/8-MCK-cynomolgusGAA-treated animals, cynomolgus GAA-protein levels also increased in all tissues examined. In the two early death animals, the female (4501) showed an ˜33-fold higher GAA-activity in the heart whereas the male (4003) had a ˜61-fold higher GAA-activity level in the heart compared to vehicle controls. Neither T-cell mediated anti-AAV8/GAA nor total anti-GAA antibodies appeared to negatively affect GAA protein or tissue enzyme activity levels.

Doses ≥2×10¹⁴ vg/kg resulted in clinical, functional and microscopic changes in the heart. Troponin-I and BNP at doses ≥2×10¹⁴ vg/kg were consistent with myocardial injury. Changes in ALT and AST were considered related to muscle myofiber degeneration/regeneration in the periphery and not related to the liver. Echocardiography showed cardiac function was adversely impaired at doses ≥2.0×10¹⁴ vg/kg and contributed to the early termination of two animals in the high dose group (5.0×10¹⁴ vg/kg), a female at Day 79 and male at Day 82. Dose dependent minimal to moderate microscopic changes were observed in the heart, skeletal muscle, and smooth muscle consisting of mixed cell interstitial inflammation characterized by variable numbers of lymphocytes, plasma cells, histiocytes with interstitial amphophilic granular material and occasional degeneration of myofibers. Additional histologic alterations generally observed in both the early deaths and main study phase animals consisted of mixed cell inflammation in the liver (males at ≥0.6×10¹⁴ vg/kg/day and females at ≥2×10¹⁴ vg/kg/day); mixed cell inflammation and edema in the gallbladder of one female given 5×10¹⁴ vg/kg/day; peri-organ mixed cell inflammation and occasional degeneration of brown fat cells at generally ≥2×10¹⁴ vg/kg/day, for both males and females (esophagus, thyroid, sternum, thymus, heart, kidney, and aorta), with higher incidence and severity in the main study animals; and dose-independent gliosis with rare neuronal degeneration in dorsal root ganglia in males and females.

In summary, the low dose (0.6×10¹⁴ vg/kg) of AAV2/8-MCK-GAA was well tolerated, whereas doses ≥2×10¹⁴ vg/kg were consistent with myocardial injury and accompanying mixed cell inflammation in muscles with occasional myofiber degeneration but also liver, adipose tissue, and dorsal root ganglia. An AAV2/8-MCK-GAA dose of 0.6×10¹⁴ vg/kg was defined as the no adverse effect limit (NOAEL).

### Example 3. Treatment of Pompe Disease in Human Patients by Administration of AAV-GAA Vectors in Accordance with a Dosing Regimen of the Disclosure

Using the compositions and methods of the disclosure, a patient having a glycogen storage disorder (e.g., Pompe disease) may be administered an AAV vector containing a transgene encoding GAA in an amount of from about 1×10¹³ vg/kg to about 3×10¹⁴ vg/kg. For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg. Administration of the vector to the patient in such a quantity can achieve the beneficial effect of augmenting GAA expression in the patient, e.g., to within 50% or 200% of wild-type levels, without inducing toxic side effects.

For example, in some embodiments, the AAV vector is administered to the patient in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the AAV vector is administered to the patient in an amount of from about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

Additionally or alternatively, a human patient having a glycogen storage disorder (e.g., Pompe disease) may be administered an agent that promotes expression of GAA in an amount sufficient to stimulate the GAA expression observed when a human subject of the same gender and similar body mass index is administered an AAV2/8 vector containing a GAA transgene under the control of a MCK promoter. For example, the agent may be administered to the patient in an amount sufficient to achieve a level of GAA activity in the patient that is equivalent to a level of GAA activity observed in the human subject of the same gender and similar body mass index upon administration to the subject of the AAV vector in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg.

For example, in some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 2×10¹³ vg/kg to about 2×10¹⁴ vg/kg, such as an in amount of about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

To assess the patient's GAA expression level following administration of a therapeutic agent described above, a physician of skill in the art may analyze one or more of the following events: (1) production of an RNA template from a DNA sequence encoding GAA; (2) processing of an RNA transcript encoding GAA protein (e.g., by splicing, editing, 5′ cap formation, and/or 3′ end processing); (3) translation of an RNA into a GAA polypeptide or protein; and (4) post-translational modification of a GAA polypeptide or protein. Expression of GAA may be assessed, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the GAA protein in a sample obtained from the subject.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating Pompe disease in a human patient in need thereof, the method comprising administering to the patient an adeno-associated viral (AAV) vector comprising a transgene encoding acid alpha-glucosidase (GAA), wherein the AAV vector is administered to the patient in an amount of from about 1×10¹³ vg/kg to about 3×10¹⁴ vg/kg.

2. A method of improving muscle function in a human patient diagnosed as having Pompe disease, the method comprising administering to the patient an AAV vector comprising a transgene encoding GAA, wherein the AAV vector is administered to the patient in an amount of from about 1×10¹³ vg/kg to about 3×10¹⁴ vg/kg.

3. A method of reducing glycogen accumulation in a human patient diagnosed as having Pompe disease, the method comprising administering to the patient an AAV vector comprising a transgene encoding GAA, wherein the AAV vector is administered to the patient in an amount of from about 1×10¹³ vg/kg to about 3×10¹⁴ vg/kg.

4. The method of claim 3, wherein administration of the AAV vector to the patient reduces glycogen accumulation in muscle tissue and/or in neuronal tissue.

5. A method of improving pulmonary function in a human patient diagnosed as having Pompe disease, the method comprising administering to the patient an AAV vector comprising a transgene encoding GAA, wherein the AAV vector is administered to the patient in an amount of from about 1×10¹³ vg/kg to about 3×10¹⁴ vg/kg.

6. A method of increasing GAA expression in a human patient diagnosed as having Pompe disease, the method comprising administering to the patient an AAV vector comprising a transgene encoding GAA, wherein the AAV vector is administered to the patient in an amount of from about 1×10¹³ vg/kg to about 3×10¹⁴ vg/kg.

7. The method of any one of claims 1-6, wherein the AAV vector is administered to the patient in an amount of from about 2×10¹³ vg/kg to about 2×10¹⁴ vg/kg.

8. The method of claim 7, wherein the AAV vector is administered to the patient in an amount of from about 3×10¹³ vg/kg to about 2×10¹⁴ vg/kg.

9. The method of claim 8, wherein the AAV vector is administered to the patient in an amount of from about 4×10¹³ vg/kg to about 2×10¹⁴ vg/kg.

10. The method of claim 9, wherein the AAV vector is administered to the patient in an amount of from about 5×10¹³ vg/kg to about 2×10¹⁴ vg/kg.

11. The method of claim 10, wherein the AAV vector is administered to the patient in an amount of from about 6×10¹³ vg/kg to about 2×10¹⁴ vg/kg.

12. The method of claim 11, wherein the AAV vector is administered to the patient in an amount of from about 7×10¹³ vg/kg to about 2×10¹⁴ vg/kg.

13. The method of claim 12, wherein the AAV vector is administered to the patient in an amount of from about 8×10¹³ vg/kg to about 2×10¹⁴ vg/kg.

14. The method of claim 13, wherein the AAV vector is administered to the patient in an amount of from about 9×10¹³ vg/kg to about 2×10¹⁴ vg/kg.

15. The method of claim 14, wherein the AAV vector is administered to the patient in an amount of from about 1×10¹⁴ vg/kg to about 2×10¹⁴ vg/kg.

16. The method of any one of claims 1-6, wherein the AAV vector is administered to the patient in an amount of about 3×10¹³ vg/kg.

17. The method of any one of claims 1-6, wherein the AAV vector is administered to the patient in an amount of about 4×10¹³ vg/kg.

18. The method of any one of claims 1-6, wherein the AAV vector is administered to the patient in an amount of about 5×10¹³ vg/kg.

19. The method of any one of claims 1-6, wherein the AAV vector is administered to the patient in an amount of about 6×10¹³ vg/kg.

20. The method of any one of claims 1-6, wherein the AAV vector is administered to the patient in an amount of about 7×10¹³ vg/kg.

21. The method of any one of claims 1-6, wherein the AAV vector is administered to the patient in an amount of about 8×10¹³ vg/kg.

22. The method of any one of claims 1-6, wherein the AAV vector is administered to the patient in an amount of about 9×10¹³ vg/kg.

23. The method of any one of claims 1-6, wherein the AAV vector is administered to the patient in an amount of about 1×10¹⁴ vg/kg.

24. The method of any one of claims 1-6, wherein the AAV vector is administered to the patient in an amount of about 1.1×10¹⁴ vg/kg.

25. The method of any one of claims 1-6, wherein the AAV vector is administered to the patient in an amount of about 1.2×10¹⁴ vg/kg.

26. The method of any one of claims 1-6, wherein the AAV vector is administered to the patient in an amount of about 1.3×10¹⁴ vg/kg.

27. The method of any one of claims 1-6, wherein the AAV vector is administered to the patient in an amount of about 1.4×10¹⁴ vg/kg.

28. The method of any one of claims 1-6, wherein the AAV vector is administered to the patient in an amount of about 1.5×10¹⁴ vg/kg.

29. The method of any one of claims 1-6, wherein the AAV vector is administered to the patient in an amount of about 1.6×10¹⁴ vg/kg.

30. The method of any one of claims 1-6, wherein the AAV vector is administered to the patient in an amount of about 1.7×10¹⁴ vg/kg.

31. The method of any one of claims 1-6, wherein the AAV vector is administered to the patient in an amount of about 1.8×10¹⁴ vg/kg.

32. The method of any one of claims 1-6, wherein the AAV vector is administered to the patient in an amount of about 1.9×10¹⁴ vg/kg.

33. The method of any one of claims 1-6, wherein the AAV vector is administered to the patient in an amount of about 2×10¹⁴ vg/kg.

34. The method of any one of claims 1-33, wherein the AAV vector is administered to the patient in a single dose comprising the amount.

35. The method of any one of claims 1-33, wherein the AAV vector is administered to the patient in two or more doses that, together, comprise the amount.

36. The method of claim 35, wherein the AAV vector is administered to the patient in from two doses to ten doses that, together, comprise the amount.

37. The method of claim 36, wherein the AAV vector is administered to the patient in two, three, or four doses that, together, comprise the amount.

38. The method of claim 37, wherein the AAV vector is administered to the patient in two doses that, together, comprise the amount.

39. The method of any one of claims 35-38, wherein the two or more doses are separated from one another by one year or more.

40. The method of any one of claims 35-38, wherein the two or more doses are administered to the patient within about 12 months of one another.

41. The method of claim 40, wherein the two or more doses are administered to the patient within from about one week to about 48 weeks of one another.

42. The method of claim 41, wherein the two or more doses are administered to the patient within from about two weeks to about 44 weeks of one another.

43. The method of claim 42, wherein the two or more doses are administered to the patient within from about three weeks to about 40 weeks of one another.

44. The method of claim 43, wherein the two or more doses are administered to the patient within from about four weeks to about 36 weeks of one another.

45. The method of claim 44, wherein the two or more doses are administered to the patient within from about five weeks to about 32 weeks of one another.

46. The method of claim 45, wherein the two or more doses are administered to the patient within from about six weeks to about 24 weeks of one another.

47. The method of claim 46, wherein the two or more doses are administered to the patient within from about 12 weeks to about 20 weeks of one another.

48. The method of claim 47, wherein the two or more doses are administered to the patient within about 13 weeks, 14 weeks, 15 weeks, 16 weeks, 17 weeks, 18 weeks, or 19 weeks of one another.

49. The method of any one of claims 1-33, wherein the AAV vector is administered to the patient in two or more doses that each, individually, comprise the amount.

50. The method of claim 49, wherein the AAV vector is administered to the patient in from two doses to ten doses that each, individually, comprise the amount.

51. The method of claim 50, wherein the AAV vector is administered to the patient in two, three, or four doses that each, individually, comprise the amount.

52. The method of claim 51, wherein the AAV vector is administered to the patient in two doses that each, individually, comprise the amount.

53. The method of any one of claims 49-52, wherein the two or more doses are separated from one another by one year or more.

54. The method of any one of claims 49-52, wherein the two or more doses are administered to the patient within about 12 months of one another.

55. The method of claim 54, wherein the two or more doses are administered to the patient within from about one week to about 48 weeks of one another.

56. The method of claim 55, wherein the two or more doses are administered to the patient within from about two weeks to about 44 weeks of one another.

57. The method of claim 56, wherein the two or more doses are administered to the patient within from about three weeks to about 40 weeks of one another.

58. The method of claim 57, wherein the two or more doses are administered to the patient within from about four weeks to about 36 weeks of one another.

59. The method of claim 58, wherein the two or more doses are administered to the patient within from about five weeks to about 32 weeks of one another.

60. The method of claim 59, wherein the two or more doses are administered to the patient within from about six weeks to about 24 weeks of one another.

61. The method of claim 60, wherein the two or more doses are administered to the patient within from about 12 weeks to about 20 weeks of one another.

62. The method of claim 61, wherein the two or more doses are administered to the patient within about 13 weeks, 14 weeks, 15 weeks, 16 weeks, 17 weeks, 18 weeks, or 19 weeks of one another.

63. The method of any one of claims 1-62, wherein the AAV vector is administered to the patient by way of intravenous, intrathecal, intracisternal, intracerebroventricular, intramuscular, intradermal, transdermal, parenteral, intranasal, subcutaneous, percutaneous, intratracheal, intraperitoneal, intraarterial, intravascular, inhalation, perfusion, lavage, and/or oral administration.

64. The method of claim 63, wherein the AAV vector is administered to the patient by way of intravenous, intrathecal, intracisternal, intracerebroventricular, and/or intramuscular administration.

65. The method of claim 64, wherein the AAV vector is administered to the patient by way of intravenous and/or intrathecal administration.

66. The method of claim 65, wherein the AAV vector is administered to the patient by way of intravenous administration.

67. The method of any one of claims 1-66, wherein the AAV is an AAV1, AAV2, AAV3, AAV4, AAV5, AAV6, AAV7, AAV8, AAV9, AAVrh74, AAVrh.8, or AAVrh.10 serotype.

68. The method of claim 67, wherein the AAV is a pseudotyped AAV.

69. The method of claim 68, wherein the pseudotyped AAV is AAV2/8.

70. The method of claim 69, wherein the pseudotyped AAV is AAV2/9.

71. The method of any one of claims 1-66, wherein the AAV comprises a recombinant capsid protein.

72. The method of any one of claims 1-71, wherein the transgene encoding GAA is operably linked to a promoter that induces expression of the transgene in a muscle and/or neuronal cell.

73. The method of claim 72, wherein the promoter is a muscle creatine kinase (MCK) promoter, desmin promoter, chicken beta actin promoter, cytomegalovirus (CMV) promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

74. The method of claim 73, wherein the promoter is a MCK promoter.

75. The method of claim 74, wherein the MCK promoter has a nucleic acid sequence that is at least 85% identical to SEQ ID NO: 1.

76. The method of claim 75, wherein the MCK promoter has a nucleic acid sequence that is at least 90% identical to SEQ ID NO: 1.

77. The method of claim 76, wherein the MCK promoter has a nucleic acid sequence that is at least 95% identical to SEQ ID NO: 1.

78. The method of claim 77, wherein the MCK promoter has a nucleic acid sequence that is at least 97% identical to SEQ ID NO: 1.

79. The method of claim 78, wherein the MCK promoter has a nucleic acid sequence that is at least 98% identical to SEQ ID NO: 1.

80. The method of claim 79, wherein the MCK promoter has a nucleic acid sequence that is at least 99% identical to SEQ ID NO: 1.

81. The method of claim 80, wherein the MCK promoter has a nucleic acid sequence that is at least 99% identical to SEQ ID NO: 1.

82. The method of claim 81, wherein the MCK promoter has a nucleic acid sequence that is 100% identical to SEQ ID NO: 1.

83. The method of any one of claims 1-82, wherein the transgene encoding GAA is operably linked to an enhancer that induces expression of the transgene in a muscle and/or neuronal cell.

84. The method of claim 83, wherein the enhancer is a CMV enhancer, a myocyte enhancer factor 2 (MEF2) enhancer, or a MyoD enhancer.

85. The method of any one of claims 1-84, wherein the GAA has an amino acid sequence that is at least 85% identical to SEQ ID NO: 2.

86. The method of claim 85, wherein the GAA has an amino acid sequence that is at least 90% identical to SEQ ID NO: 2.

87. The method of claim 86, wherein the GAA has an amino acid sequence that is at least 95% identical to SEQ ID NO: 2.

88. The method of claim 87, wherein the GAA has an amino acid sequence that is at least 97% identical to SEQ ID NO: 2.

89. The method of claim 88, wherein the GAA has an amino acid sequence that is at least 98% identical to SEQ ID NO: 2.

90. The method of claim 89, wherein the GAA has an amino acid sequence that is at least 99% identical to SEQ ID NO: 2.

91. The method of claim 90, wherein the GAA has an amino acid sequence that is 100% identical to SEQ ID NO: 2.

92. The method of any one of claims 1-91, wherein the patient has infantile-onset Pompe disease.

93. The method of claim 92, wherein the patient is from about one month to about one year of age.

94. The method of claim 93, wherein the patient is from about one month to about six months of age.

95. The method of any one of claims 92-94, wherein, prior to administration of the AAV vector to the patient, the patient exhibits a symptom selected from feeding difficulties, failure to thrive, hypotonia, progressive weakness, respiratory distress, severe enlargement of the tongue, and thickening of the heart muscle.

96. The method of any one of claims 1-91, wherein the patient has late-onset Pompe disease.

97. The method of claim 96, wherein the patient exhibits endogenous GAA activity of from about 1% to about 40% of the endogenous GAA activity of a human of the same gender and similar body mass index that does not have Pompe disease.

98. The method of any one of claims 1-97, wherein the patient has not previously received GAA enzyme replacement therapy.

99. The method of any one of claims 1-98, wherein the patient has previously received GAA enzyme replacement therapy.

100. The method of any one of claims 1-99, wherein, following administration of the AAV vector to the patient, the patient exhibits endogenous GAA activity of from about 50% to about 200% of the endogenous GAA activity of a human of the same gender and similar body mass index that does not have Pompe disease.

101. The method of any one of claims 1-100, wherein, following administration of the AAV vector to the patient, the patient exhibits a reduction in glycogen in skeletal muscle, cardiac muscle, and/or neuronal tissue.

102. A method of treating Pompe disease in a human patient in need thereof, the method comprising administering to the patient an agent that increases GAA expression, wherein the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector comprising a transgene encoding GAA in an amount of from about 1×10¹³ vg/kg to about 3×10¹⁴ vg/kg, wherein the transgene encoding GAA is operably linked to a MCK promoter.

103. A method of improving muscle function in a human patient diagnosed as having Pompe disease, the method comprising administering to the patient an agent that increases GAA expression, wherein the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector comprising a transgene encoding GAA in an amount of from about 1×10¹³ vg/kg to about 3×10¹⁴ vg/kg, wherein the transgene encoding GAA is operably linked to a MCK promoter.

104. A method of reducing glycogen accumulation in a human patient diagnosed as having Pompe disease, the method comprising administering to the patient an agent that increases GAA expression, wherein the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector comprising a transgene encoding GAA in an amount of from about 1×10¹³ vg/kg to about 3×10¹⁴ vg/kg, wherein the transgene encoding GAA is operably linked to a MCK promoter.

105. The method of claim 104, wherein administration of the agent to the patient reduces glycogen accumulation in muscle tissue and/or in neuronal tissue.

106. A method of improving pulmonary function in a human patient diagnosed as having Pompe disease, the method comprising administering to the patient an agent that increases GAA expression, wherein the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector comprising a transgene encoding GAA in an amount of from about 1×10¹³ vg/kg to about 3×10¹⁴ vg/kg, wherein the transgene encoding GAA is operably linked to a MCK promoter.

107. A method of increasing GAA expression in a human patient diagnosed as having Pompe disease, the method comprising administering to the patient an agent that increases GAA expression, wherein the agent is administered to the patient in an amount sufficient to achieve a level of GAA activity in the patient that is equivalent to a level of GAA activity observed in a human subject of the same gender and similar body mass index as the patient upon administration to the subject of an AAV2/8 vector comprising a transgene encoding GAA in an amount of from about 1×10¹³ vg/kg to about 3×10¹⁴ vg/kg, wherein the transgene encoding GAA is operably linked to a MCK promoter.

108. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 2×10¹³ vg/kg to about 2×10¹⁴ vg/kg.

109. The method of claim 108, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 3×10¹³ vg/kg to about 2×10¹⁴ vg/kg.

110. The method of claim 109, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 4×10¹³ vg/kg to about 2×10¹⁴ vg/kg.

111. The method of claim 110, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 5×10¹³ vg/kg to about 2×10¹⁴ vg/kg.

112. The method of claim 111, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 6×10¹³ vg/kg to about 2×10¹⁴ vg/kg.

113. The method of claim 112, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 7×10¹³ vg/kg to about 2×10¹⁴ vg/kg.

114. The method of claim 113, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 8×10¹³ vg/kg to about 2×10¹⁴ vg/kg.

115. The method of claim 114, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 9×10¹³ vg/kg to about 2×10¹⁴ vg/kg.

116. The method of claim 115, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of from about 1×10¹⁴ vg/kg to about 2×10¹⁴ vg/kg.

117. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 3×10¹³ vg/kg.

118. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 4×10¹³ vg/kg.

119. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 5×10¹³ vg/kg.

120. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 6×10¹³ vg/kg.

121. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 7×10¹³ vg/kg.

122. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 8×10¹³ vg/kg.

123. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 9×10¹³ vg/kg.

124. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1×10¹⁴ vg/kg.

125. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.1×10¹⁴ vg/kg.

126. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.2×10¹⁴ vg/kg.

127. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.3×10¹⁴ vg/kg.

128. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.4×10¹⁴ vg/kg.

129. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.5×10¹⁴ vg/kg.

130. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.6×10¹⁴ vg/kg.

131. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.7×10¹⁴ vg/kg.

132. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.8×10¹⁴ vg/kg.

133. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 1.9×10¹⁴ vg/kg.

134. The method of any one of claims 102-107, wherein the agent is administered to the patient in an amount sufficient to achieve a level of GAA activity in the patient that is equivalent to a level of GAA activity observed in the human subject of the same gender and similar body mass index as the patient upon administration to the subject of the AAV vector in an amount of about 2×10¹⁴ vg/kg.

135. The method of any one of claims 102-134, wherein the agent is administered to the patient in a single dose.

136. The method of any one of claims 102-134, wherein the agent is administered to the patient in two or more doses.

137. The method of any one of claims 102-136, wherein the agent is administered to the patient by way of intravenous, intrathecal, intracisternal, intracerebroventricular, intramuscular, intradermal, transdermal, parenteral, intranasal, subcutaneous, percutaneous, intratracheal, intraperitoneal, intraarterial, intravascular, inhalation, perfusion, lavage, and/or oral administration.

138. The method of any one of claims 102-137, wherein the agent comprises (i) a nucleic acid molecule encoding GAA, (ii) one or more interfering RNA molecules that collectively increase expression of endogenous GAA, (iii) one or more nucleic acid molecules encoding the one or more interfering RNA molecules, (iv) a GAA protein, and/or (v) one or more small molecules that collectively increase expression of endogenous GAA.

139. The method of claim 138, wherein the one or more interfering RNA molecules comprise short interfering RNA (siRNA), short hairpin RNA (shRNA), and/or micro RNA (miRNA).

140. The method of claim 139, wherein the agent comprises a nucleic acid molecule encoding GAA.

141. The method of claim 140, wherein the nucleic acid molecule is provided to the patient by administering to the patient a viral vector that comprises the nucleic acid molecule.

142. The method of claim 141, wherein the viral vector is an AAV, an adenovirus, a parvovirus, a coronavirus, a rhabdovirus, a paramyxovirus, a picornavirus, an alphavirus, a herpes virus, a poxvirus, or a Retroviridae family virus.

143. The method of claim 142, wherein the viral vector is an AAV.

144. The method of claim 143, wherein the AAV has a serotype selected from AAV1, AAV2, AAV3, AAV4, AAV5, AAV6, AAV7, AAV8, AAV9, AAV10, and AAVrh74.

145. The method of claim 143, wherein the AAV is a pseudotyped AAV.

146. The method of claim 145, wherein the pseudotyped AAV is AAV2/8.

147. The method of claim 145, wherein the pseudotyped AAV is AAV2/9.

148. The method of claim 143, wherein the AAV comprises a recombinant capsid protein.

149. The method of any one of claims 138-148, wherein the nucleic acid molecule encoding GAA is operably linked to a promoter that induces expression of the transgene in a muscle and/or neuronal cell.

150. The method of claim 149, wherein the promoter is a muscle MCK promoter, desmin promoter, chicken beta actin promoter, CMV promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

151. The method of any one of claims 138-150, wherein the nucleic acid molecule encoding GAA is operably linked to an enhancer that induces expression of the transgene in a muscle and/or neuronal cell.

152. The method of claim 151, wherein the enhancer is a CMV enhancer, a MEF2 enhancer, or a MyoD enhancer.

153. The method of any one of claims 102-152, wherein the GAA has an amino acid sequence that is at least 85% identical to SEQ ID NO: 2.

154. The method of claim 153, wherein the GAA has an amino acid sequence that is at least 90% identical to SEQ ID NO: 2.

155. The method of claim 154, wherein the GAA has an amino acid sequence that is at least 95% identical to SEQ ID NO: 2.

156. The method of claim 155, wherein the GAA has an amino acid sequence that is at least 97% identical to SEQ ID NO: 2.

157. The method of claim 156, wherein the GAA has an amino acid sequence that is at least 98% identical to SEQ ID NO: 2.

158. The method of claim 157, wherein the GAA has an amino acid sequence that is at least 99% identical to SEQ ID NO: 2.

159. The method of claim 158, wherein the GAA has an amino acid sequence that is 100% identical to SEQ ID NO: 2.

160. The method of any one of claims 102-159, wherein the patient has infantile-onset Pompe disease.

161. The method of claim 160, wherein the patient is from about one month to about one year of age.

162. The method of claim 161, wherein the patient is from about one month to about six months of age.

163. The method of any one of claims 160-162, wherein, prior to administration of the AAV vector to the patient, the patient exhibits a symptom selected from feeding difficulties, failure to thrive, hypotonia, progressive weakness, respiratory distress, severe enlargement of the tongue, and thickening of the heart muscle.

164. The method of any one of claims 102-159, wherein the patient has late-onset Pompe disease.

165. The method of claim 164, wherein the patient exhibits endogenous GAA activity of from about 1% to about 40% of the endogenous GAA activity of a human of the same gender and similar body mass index that does not have Pompe disease.

166. The method of any one of claims 102-165, wherein the patient has not previously received GAA enzyme replacement therapy.

167. The method of any one of claims 102-166, wherein the patient has previously received GAA enzyme replacement therapy.

168. The method of any one of claims 102-167, wherein, following administration of the AAV vector to the patient, the patient exhibits endogenous GAA activity of from about 50% to about 200% of the endogenous GAA activity of a human of the same gender and similar body mass index that does not have Pompe disease.

169. The method of any one of claims 102-168, wherein, following administration of the AAV vector to the patient, the patient exhibits a reduction in glycogen in skeletal muscle, cardiac muscle, and/or neuronal tissue.

170. A kit comprising an AAV vector comprising a transgene encoding GAA and a package insert, wherein the package insert instructs a user of the kit to administer the AAV vector a human patient in accordance with the method of any one of claims 1-101.

171. A kit comprising an agent that increases GAA expression and a package insert, wherein the package insert instructs a user of the kit to administer the agent to a human patient in accordance with the method of any one of claims 102-169.

172. Use of an AAV vector comprising a transgene encoding GAA in the manufacture of a medicament for treating Pompe disease in a human patient in need thereof, wherein the medicament comprises the AAV vector in an amount of from about 1×10¹³ vg/kg to about 3×10¹⁴ vg/kg.

173. Use of an AAV vector comprising a transgene encoding GAA in the manufacture of a medicament for improving muscle function in a human patient diagnosed as having Pompe disease, wherein the medicament comprises the AAV vector in an amount of from about 1×10¹³ vg/kg to about 3×10¹⁴ vg/kg.

174. Use of an AAV vector comprising a transgene encoding GAA in the manufacture of a medicament for reducing glycogen accumulation in a human patient diagnosed as having Pompe disease, wherein the medicament comprises the AAV vector in an amount of from about 1×10¹³ vg/kg to about 3×10¹⁴ vg/kg.

175. The use of claim 174, wherein administration of the AAV vector to the patient reduces glycogen accumulation in muscle tissue and/or in neuronal tissue.

176. Use of an AAV vector comprising a transgene encoding GAA in the manufacture of a medicament for improving pulmonary function in a human patient diagnosed as having Pompe disease, wherein the medicament comprises the AAV vector in an amount of from about 1×10¹³ vg/kg to about 3×10¹⁴ vg/kg.

177. Use of an AAV vector comprising a transgene encoding GAA in the manufacture of a medicament for increasing GAA expression in a human patient diagnosed as having Pompe disease, wherein the medicament comprises the AAV vector in an amount of from about 1×10¹³ vg/kg to about 3×10¹⁴ vg/kg.

178. The use of any one of claims 172-177, wherein the medicament comprises the AAV vector in an amount of from about 2×10¹³ vg/kg to about 2×10¹⁴ vg/kg.

179. The use of claim 178, wherein the medicament comprises the AAV vector in an amount of from about 3×10¹³ vg/kg to about 2×10¹⁴ vg/kg.

180. The use of claim 179, wherein the medicament comprises the AAV vector in an amount of from about 4×10¹³ vg/kg to about 2×10¹⁴ vg/kg.

181. The use of claim 180, wherein the medicament comprises the AAV vector in an amount of from about 5×10¹³ vg/kg to about 2×10¹⁴ vg/kg.

182. The use of claim 181, wherein the medicament comprises the AAV vector in an amount of from about 6×10¹³ vg/kg to about 2×10¹⁴ vg/kg.

183. The use of claim 182, wherein the medicament comprises the AAV vector in an amount of from about 7×10¹³ vg/kg to about 2×10¹⁴ vg/kg.

184. The use of claim 183, wherein the medicament comprises the AAV vector in an amount of from about 8×10¹³ vg/kg to about 2×10¹⁴ vg/kg.

185. The use of claim 184, wherein the medicament comprises the AAV vector in an amount of from about 9×10¹³ vg/kg to about 2×10¹⁴ vg/kg.

186. The use of claim 185, wherein the medicament comprises the AAV vector in an amount of from about 1×10¹⁴ vg/kg to about 2×10¹⁴ vg/kg.

187. The use of any one of claims 172-177, wherein the medicament comprises the AAV vector in an amount of about 3×10¹³ vg/kg.

188. The use of any one of claims 172-177, wherein the medicament comprises the AAV vector in an amount of about 4×10¹³ vg/kg.

189. The use of any one of claims 172-177, wherein the medicament comprises the AAV vector in an amount of about 5×10¹³ vg/kg.

190. The use of any one of claims 172-177, wherein the medicament comprises the AAV vector in an amount of about 6×10¹³ vg/kg.

191. The use of any one of claims 172-177, wherein the medicament comprises the AAV vector in an amount of about 7×10¹³ vg/kg.

192. The use of any one of claims 172-177, wherein the medicament comprises the AAV vector in an amount of about 8×10¹³ vg/kg.

193. The use of any one of claims 172-177, wherein the medicament comprises the AAV vector in an amount of about 9×10¹³ vg/kg.

194. The use of any one of claims 172-177, wherein the medicament comprises the AAV vector in an amount of about 1×10¹⁴ vg/kg.

195. The use of any one of claims 172-177, wherein the medicament comprises the AAV vector in an amount of about 1.1×10¹⁴ vg/kg.

196. The use of any one of claims 172-177, wherein the medicament comprises the AAV vector in an amount of about 1.2×10¹⁴ vg/kg.

197. The use of any one of claims 172-177, wherein the medicament comprises the AAV vector in an amount of about 1.3×10¹⁴ vg/kg.

198. The use of any one of claims 172-177, wherein the medicament comprises the AAV vector in an amount of about 1.4×10¹⁴ vg/kg.

199. The use of any one of claims 172-177, wherein the medicament comprises the AAV vector in an amount of about 1.5×10¹⁴ vg/kg.

200. The use of any one of claims 172-177, wherein the medicament comprises the AAV vector in an amount of about 1.6×10¹⁴ vg/kg.

201. The use of any one of claims 172-177, wherein the medicament comprises the AAV vector in an amount of about 1.7×10¹⁴ vg/kg.

202. The use of any one of claims 172-177, wherein the medicament comprises the AAV vector in an amount of about 1.8×10¹⁴ vg/kg.

203. The use of any one of claims 172-177, wherein the medicament comprises the AAV vector in an amount of about 1.9×10¹⁴ vg/kg.

204. The use of any one of claims 172-177, wherein the medicament comprises the AAV vector in an amount of about 2×10¹⁴ vg/kg.

205. The use of any one of claims 172-204, wherein the AAV vector is formulated for administration to the patient in a single dose comprising the amount.

206. The use of any one of claims 172-204, wherein the AAV vector is formulated for administration to the patient in two or more doses that, together, comprise the amount.

207. The use of claim 206, wherein the AAV vector is formulated for administration to the patient in from two doses to ten doses that, together, comprise the amount.

208. The use of claim 207, wherein the AAV vector is formulated for administration to the patient in two, three, or four doses that, together, comprise the amount.

209. The use of claim 208, wherein the AAV vector is formulated for administration to the patient in two doses that, together, comprise the amount.

210. The use of any one of claims 172-204, wherein the AAV vector is formulated for administration to the patient in two or more doses that each, individually, comprise the amount.

211. The use of claim 210, wherein the AAV vector is formulated for administration to the patient in in from two doses to ten doses that each, individually, comprise the amount.

212. The use of claim 211, wherein the AAV vector is formulated for administration to the patient in two, three, or four doses that each, individually, comprise the amount.

213. The use of claim 212, wherein the AAV vector is formulated for administration to the patient in two doses that each, individually, comprise the amount.

214. The use of any one of claims 172-213, wherein the AAV vector is formulated for intravenous, intrathecal, intracisternal, intracerebroventricular, intramuscular, intradermal, transdermal, parenteral, intranasal, subcutaneous, percutaneous, intratracheal, intraperitoneal, intraarterial, intravascular, inhalation, perfusion, lavage, and/or oral administration to the patient.

215. The use of claim 214, wherein the AAV vector is formulated for intravenous, intrathecal, intracisternal, intracerebroventricular, and/or intramuscular administration to the patient.

216. The use of claim 215, wherein the AAV vector is formulated for intravenous and/or intrathecal administration to the patient.

217. The use of claim 216, wherein the AAV vector is formulated for intravenous administration to the patient.

218. The use of any one of claims 172-217, wherein the AAV is an AAV1, AAV2, AAV3, AAV4, AAV5, AAV6, AAV7, AAV8, AAV9, AAVrh74, AAVrh.8, or AAVrh.10 serotype.

219. The use of claim 218, wherein the AAV is a pseudotyped AAV.

220. The use of claim 219, wherein the pseudotyped AAV is AAV2/8.

221. The use of claim 219, wherein the pseudotyped AAV is AAV2/9.

222. The use of any one of claims 172-217, wherein the AAV comprises a recombinant capsid protein.

223. The use of any one of claims 172-222, wherein the transgene encoding GAA is operably linked to a promoter that induces expression of the transgene in a muscle and/or neuronal cell.

224. The use of claim 223, wherein the promoter is a MCK promoter, desmin promoter, chicken beta actin promoter, CMV promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

225. The use of claim 224, wherein the promoter is a MCK promoter.

226. The use of claim 225, wherein the MCK promoter has a nucleic acid sequence that is at least 85% identical to SEQ ID NO: 1.

227. The use of claim 226, wherein the MCK promoter has a nucleic acid sequence that is at least 90% identical to SEQ ID NO: 1.

228. The use of claim 227, wherein the MCK promoter has a nucleic acid sequence that is at least 95% identical to SEQ ID NO: 1.

229. The use of claim 228, wherein the MCK promoter has a nucleic acid sequence that is at least 97% identical to SEQ ID NO: 1.

230. The use of claim 229, wherein the MCK promoter has a nucleic acid sequence that is at least 98% identical to SEQ ID NO: 1.

231. The use of claim 230, wherein the MCK promoter has a nucleic acid sequence that is at least 99% identical to SEQ ID NO: 1.

232. The use of claim 231, wherein the MCK promoter has a nucleic acid sequence that is at least 99% identical to SEQ ID NO: 1.

233. The use of claim 232, wherein the MCK promoter has a nucleic acid sequence that is 100% identical to SEQ ID NO: 1.

234. The use of any one of claims 172-233, wherein the transgene encoding GAA is operably linked to an enhancer that induces expression of the transgene in a muscle and/or neuronal cell.

235. The use of claim 234, wherein the enhancer is a CMV enhancer, a MEF2 enhancer, or a MyoD enhancer.

236. The use of any one of claims 172-235, wherein the GAA has an amino acid sequence that is at least 85% identical to SEQ ID NO: 2.

237. The use of claim 236, wherein the GAA has an amino acid sequence that is at least 90% identical to SEQ ID NO: 2.

238. The use of claim 237, wherein the GAA has an amino acid sequence that is at least 95% identical to SEQ ID NO: 2.

239. The use of claim 238, wherein the GAA has an amino acid sequence that is at least 97% identical to SEQ ID NO: 2.

240. The use of claim 239, wherein the GAA has an amino acid sequence that is at least 98% identical to SEQ ID NO: 2.

241. The use of claim 240, wherein the GAA has an amino acid sequence that is at least 99% identical to SEQ ID NO: 2.

242. The use of claim 241, wherein the GAA has an amino acid sequence that is 100% identical to SEQ ID NO: 2.

243. The use of any one of claims 172-242, wherein the patient has infantile-onset Pompe disease.

244. The use of claim 243, wherein the patient is from about one month to about one year of age.

245. The use of claim 244, wherein the patient is from about one month to about six months of age.

246. The use of any one of claims 242-245, wherein, prior to administration of the AAV vector to the patient, the patient exhibits a symptom selected from feeding difficulties, failure to thrive, hypotonia, progressive weakness, respiratory distress, severe enlargement of the tongue, and thickening of the heart muscle.

247. The use of any one of claims 172-242, wherein the patient has late-onset Pompe disease.

248. The use of claim 247, wherein the patient exhibits endogenous GAA activity of from about 1% to about 40% of the endogenous GAA activity of a human of the same gender and similar body mass index that does not have Pompe disease.

249. The use of any one of claims 172-248, wherein the patient has not previously received GAA enzyme replacement therapy.

250. The use of any one of claims 172-248, wherein the patient has previously received GAA enzyme replacement therapy.

251. The use of any one of claims 172-250, wherein, following administration of the AAV vector to the patient, the patient exhibits endogenous GAA activity of from about 50% to about 200% of the endogenous GAA activity of a human of the same gender and similar body mass index that does not have Pompe disease.

252. The use of any one of claims 172-251, wherein, following administration of the AAV vector to the patient, the patient exhibits a reduction in glycogen in skeletal muscle, cardiac muscle, and/or neuronal tissue.


SEQUENCE LISTING (3 sequences)

SEQ ID NO: 1 - DNA, 584 bp, Homo sapiens
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 2 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 3 - DNA, 2859 bp, Homo sapiens
atgggggtgaggcaccccccctgcagccacaggctgctggctgtgtgtgccctggtcagcctggccactgctgccctgctgggccacatcctgctgcatgacttcctgctggtgcccagagagctgtctggcagcagccctgtgctggaggaaacccaccctgcccaccagcagggggccagcaggcctggccccagggatgcccaggcccaccctggcaggcccagggctgtgcccacccagtgtgatgtgccccccaacagcaggtttgactgtgcccctgacaaggccatcacccaggagcagtgtgaggccaggggctgctgctacatccctgccaagcagggcctgcagggggcccagatgggccagccctggtgcttcttccccccctcatacccctcctacaagctggagaacctgagcagctctgagatgggctacactgccaccctgaccaggaccaccccgacgttcttccccaaggacatcctgaccctgaggctggatgtgatgatggagactgagaacaggctgcacttcaccatcaaggaccctgccaacaggagatatgaggtgcccctggaaaccccccatgtgcacagcagggcccccagccccctgtactctgtggagttctctgaggagccctttggggtgattgtgaggaggcagctggatggcagggtgctgctgaacaccactgtggcccccctgttctttgctgaccagttcctgcagctgagcaccagcctgcccagccagtacatcactggcctggctgagcacctgagccccctgatgctgagcaccagctggaccaggatcaccctgtggaacagggacctggcccccacccctggggccaacctgtatggcagccaccccttctacctggccctggaggatgggggctctgcccatggggtgttcctgctgaacagcaatgccatggatgtggtgctgcagcccagccctgccctgagctggaggagcactgggggcatcctggatgtgtacatcttcctgggccctgagcccaagtctgtggtgcagcagtacctggatgtggtgggctaccccttcatgcccccctactggggcctgggcttccacctgtgcagatggggctacagcagcactgccatcaccaggcaggtggtggagaacatgaccagggcccacttccccctggatgtgcagtggaatgacctggactacatggacagcaggagggacttcaccttcaacaaggatggcttcagggacttccctgccatggtgcaggagctgcaccaggggggcaggagatacatgatgattgtggaccctgccatcagcagctctggccctgctggcagctacaggccctatgatgagggcctgaggaggggggtgttcatcaccaatgagactggccagcccctgattggcaaggtctggcctggcagcactgccttccctgacttcaccaaccccactgccctggcctggtgggaggacatggtggctgagttccatgaccaggtgccctttgatggcatgtggattgacatgaatgagcccagcaacttcatcaggggctctgaggatggctgccccaacaatgagctggagaaccccccctatgtgcctggggtggtggggggcaccctgcaggctgccaccatctgtgccagcagccaccagttcctgagcacccactacaacctgcacaacctgtatggcctgactgaggccattgccagccacagggccctggtgaaggccaggggcaccaggccctttgtgatcagcaggagcacctttgctggccatggcagatatgctggccactggactggggatgtgtggagcagctgggagcagctggccagctctgtgcctgagatcctgcagttcaacctgctgggggtgcccctggtgggggctgatgtgtgtggcttcctgggcaacacctctgaggagctgtgtgtgaggtggacccagctgggggccttctaccccttcatgaggaaccacaacagcctgctgagcctgccccaggagccctacagcttctctgagcctgcccagcaggccatgaggaaggccctgaccctgagatatgccctgctgccccacctgtacaccctgttccaccaggcccatgtggctggggagactgtggccaggcccctgttcctggagttccccaaggacagcagcacctggactgtggaccaccagctgctgtggggggaggccctgctgatcacccctgtgctgcaagctggcaaggctgaggtgactggctacttccccctgggcacttggtatgacctgcagactgtgcctgtggaggccctgggcagcctgccccccccccctgctgccccacgggagcctgccatccactctgagggccagtgggtgaccctgcctgcccccctggacaccatcaatgtgcacctgagggctggctacatcatccccctgcaaggccctggcctgaccaccactgagagcaggcagcagcccatggccctggctgtggccctgaccaaggggggggaggccaggggggagctgttctgggatgatggggagagcctggaggtgctggagaggggggcctacacccaggtgatcttcctggccaggaacaacaccattgtgaatgagctggtgagggtgacctctgagggggctggcctgcagctgcagaaggtcactgtgctgggggtggccactgccccccagcaggtgctgagcaatggggtgcctgtgagcaacttcacctacagccctgacaccaaggtgctggacatctgtgtgagcctgctgatgggggagcagttcctggtcagctggtgctga